(19) United States
(12) Patent Application Publication
(10) Pub. No.: US 2024/0050449 A1
(43) Pub. Date: Feb. 15, 2024

(54) Use of Acetyltanshinone IIA in Preparation of Medicament for Treating Lung Cancer and Medicament for Treating Lung Cancer

(71) Applicant: UNIVERSITY OF MACAU, Macau (CN)

(72) Inventors: Qian Luo, Macau (CN); Bin Huang, Macau (CN)

(21) Appl. No.: 18/384,952

(22) Filed: Oct. 30, 2023

Related U.S. Application Data
(63) Continuation of application No. PCT/CN2022/117849, filed on Sep. 8, 2022.

(30) Foreign Application Priority Data
Feb. 28, 2022 (CN) 202210189883.X

(51) Int. Cl. A61K 31/58; A61P 35/00

(52) U.S. Cl. CPC A61K 31/58; A61P 35/00

(57) ABSTRACT

The present disclosure provides the use of acetyltanshinone IIA in the preparation of medicament for treating lung cancer and medicament for treating lung cancer, which falls within the technical field of medicine. This protocol proposes the use of acetyltanshinone IIA in treating lung cancer, especially non-small cell lung cancer (NSCLC), which can antagonize primary and acquired drug resistance of NSCLC cells to epidermal growth factor receptor, tyrosine kinase inhibitor (EGFR TKIs), by using small molecule compound acetyltanshinone IIA. The present disclosure further identifies the molecular target of ATA and elucidates its mechanism of inhibiting the growth of drug-resistant NSCLC cells and tumors. The medicament containing acetyltanshinone IIA is expected to become a multi-target anticancer agent for treating TKI drug-resistant NSCLC.

### CROSS-REFERENCE TO RELATED APPLICATIONS

This present application is a continuation of International Application No. PCT/CN2022/117849 filed on Sep. 8, 2022, which claims the priority of the Chinese patent application with the application number CN202210189883.X and titled “ Use of Acetyltanshinone IIA in Preparation of Medicament for Treating Lung Cancer and Medicament for Treating Lung Cancer” submitted to the NATIONAL INTELLECTUAL PROPERTY ADMINISTRATION, PRC on Feb. 28, 2022, all of which are incorporated by reference in this application.

### REFERENCE TO SEQUENCE LISTING

This application includes a Sequence Listing submitted electronically as an XML file named Sequence_listing_BOSA-23006-USPT, created on Oct. 18, 2023, with a size of 24 kilobytes. The Sequence Listing is incorporated herein by reference.

### TECHNICAL FIELD

The present disclosure relates to the technical field of medicine, and in particular, to the use of acetyltanshinone IIA in the preparation of medicament for treating lung cancer and medicament for treating lung cancer.

### BACKGROUND ART

Among all types of cancer, lung cancer has the highest morbidity and mortality worldwide, with 2.1 million new cases and about 1.8 million deaths worldwide in 2018. Non-small cell lung cancer (NSCLC) accounts for 85% of all newly diagnosed lung cancers and is the major histological subtype of the disease. Activating mutations of the epidermal growth factor receptor (EGFR) are the most common driving mutations and may be targeted for the treatment of NSCLC. The development of epidermal growth factor receptor-targeted therapies has revolutionized the clinical treatment of NSCLC. Targeted therapy with an epidermal growth factor receptor tyrosine kinase inhibitor (EGFR TKIs) has improved the prognosis of NSCLC patients. However, due to primary or acquired drug resistance to EGFR TKIs, responses to these medicaments are often incomplete and transient, which has become a complex clinical problem during NSCLC treatment.

Studies have shown that various mechanisms can lead to drug resistance to EGFR TKIs. Specifically, causes of primary drug resistance to EGFR TKIs include but are not limited to, up-regulation of wild-type EGFR, activation of KRAS or BRAF mutations, Bim deletion, some rare EGFR mutations, activation of carcinoma-associated fibroblasts (CAF) or NF-κB signaling in the tumor microenvironment. In approximately 30% of NSCLC patients, an activated KRAS mutation is observed in codons 12 or 13, which is associated with drug resistance to EGFR TKIs. Acquired drug resistance to EGFR TKIs involves multiple mechanisms, including acquisition of a second mutation of T790M in EGFR with a primary mutation of L858R, sustained activation of bypass signaling pathways (such as the MET pathway or the HER2 pathway), inactivation of tumor suppressors (such as loss of PTEN or neurofibromin deletion of neurofibromin), histological transition from epithelial to SCLCs, epithelial-mesenchymal transition (EMT), or intratumoral heterogeneity. Acquired drug resistance to Erlotinib is primarily mediated by the T790M EGFR secondary mutation, which occurs in 50-65% of NSCLC patients resistant to EGFR TKIs. In addition, amplification of the MET gene is found in 5-10% of NSCLC patients with acquired drug resistance to EGFR TKIs. Therefore, new strategies and medicaments are urgently needed to overcome the primary and acquired drug resistance of NSCLC to EGFR TKIs.

### SUMMARY OF THE INVENTION

The present disclosure provides the use of acetyltanshinone IIA in the preparation of a medicament for treating lung cancer.

In an alternative embodiment, acetyltanshinone IIA is used in the manufacture of a medicament for treating non-small cell lung cancer.

The present disclosure also provides the use of acetyltanshinone IIA in the preparation of a lung cancer cell growth inhibitor.

In an alternative embodiment, acetyltanshinone IIA is used for the preparation of a non-small cell lung cancer cell growth inhibitor.

In an alternative embodiment, acetyltanshinone IIA is used to prepare an A549 cell growth inhibitor, an H358 cell growth inhibitor, an H1975 cell growth inhibitor, and/or an H1650 cell growth inhibitor.

The present disclosure also provides the use of acetyltanshinone IIA in the preparation of a protein synthesis inhibitor.

In alternative embodiments, the protein synthesis inhibitor comprises an inhibitor of cell cycle-related protein synthesis.

In an alternative embodiment, the proteins corresponding to the protein synthesis inhibitors comprise at least one of p70S6K, cyclin D3, AURKA, PLK1, cyclin B1, survivin, EGFR, and MET.

The present disclosure also provides the use of acetyltanshinone IIA in the preparation of inhibitors for phosphorylation level of signaling molecules downstream of proteins.

In an alternative embodiment, the acetyltanshinone IIA is used to prepare p70S6K and/or S6RP phosphorylation inhibitors.

The present disclosure also provides the use of acetyltanshinone IIA in the preparation of p21 transcriptional activator or p53 promoter.

The present disclosure also provides a medicament for treating lung cancer, the composition of the medicament comprising acetyltanshinone IIA.

In an alternative embodiment, the medicament is a drug for treating non-small cell lung cancer.

In an alternative embodiment, the pharmaceutical composition further comprises a lung cancer cell growth inhibitor, a protein synthesis inhibitor, an inhibitor for phosphorylation level of signaling molecules downstream of proteins, a p21 transcriptional activator, and a p53 promoter containing acetyltanshinone IIA.

The present disclosure also provides the use of acetyltanshinone IIA for treating a disease associated with lung cancer.

The present disclosure also provides the use of a medicament as described above for treating a disease associated with lung cancer.

The present disclosure also provides a method of treating a disease associated with lung cancer in a subject, comprising: administering to a subject in need thereof a medicament as described above.

In an alternative embodiment, the disease associated with lung cancer comprises non-small cell lung cancer and small cell lung cancer.

In an alternative embodiment, non-small cell lung cancer comprises adenocarcinoma, squamous cell carcinoma, and large cell carcinoma.

### BRIEF DESCRIPTION OF THE DRAWINGS

In order to more clearly illustrate the technical solutions of the embodiments of the present disclosure, a brief description will be given below of the accompanying drawings that are required to be used in the embodiments. It is to be understood that the following drawings illustrate only certain embodiments of the present disclosure and are therefore not to be considered limiting of its scope and that other related drawings may be obtained by those skilled in the art without involving any inventive effort.

FIG. 1 and FIG. 9 show the results of ATA effectively inhibiting the growth, migration, and invasion of NSCLC cells that are primary or acquired drug-resistant to EGFR TKIs in Example 1; FIG. 2 shows the result that ATA significantly reduced the protein levels of EGFR and MET in drug-resistant NSCLC cells in Example 2;

FIG. 3 and FIG. 10 show the results of ATA inhibiting the growth of drug-resistant NSCLC cells by reducing p70S6K in Example 3;

FIG. 4 and FIG. 11 show the results of ATA degradation of p70S6K protein by binding to p70S6K and increasing its ubiquitination in Example 4;

FIG. 5 and FIG. 12 show the results of ATA blocking cell cycle progression in G1/S phase by affecting p21 and cyclin D3 in Example 5;

FIG. 6 show the result of ATA inhibiting the growth of drug-resistant NSCLC cells by reducing the protein level of AURKA in Example 6;

FIG. 7 shows the result that ATA affects cell cycle-related proteins by reducing the protein level of p70S6K in Example 7, and FIG. 13 shows the comparison of protein levels between normal and drug-resistant NSCLC cells in Example 7;

FIG. 8 shows the growth results of ATA inhibiting the growth of drug-resistant NSCLC-derived xenografts tumor in mice in Example 8, FIG. 14 shows the result of weight after receiving drug treatment in Example 8, and FIG. 15 shows the results of expression levels of p70S6K and AURKA in cancer samples and normal samples of different types of cancer in Example 8.

### DETAILED DESCRIPTION OF THE INVENTION

In order that the objects, aspects, and advantages of the embodiments of the present disclosure will become more apparent, a more complete description of the embodiments of the present disclosure will be rendered by reference to the following description. Where specific conditions are not specified in the examples, they are carried out according to conventional conditions or conditions suggested by the manufacturer. The reagents or instruments used are not specified by the manufacturer and are conventional products commercially available.

The use of acetyltanshinone IIA provided in the present disclosure in the preparation of a medicament for treating lung cancer and a medicament for treating lung cancer are described below.

An embodiment of the present disclosure proposes the use of acetyltanshinone IIA in the preparation of a medicament for treating lung cancer. In some embodiments, acetyltanshinone IIA is particularly used for the preparation of a medicament for treating non-small cell lung cancer.

An embodiment of the present disclosure also provides the use of acetyltanshinone IIA in the preparation of a lung cancer cell growth inhibitor. In some embodiments, acetyltanshinone IIA is particularly useful for preparing growth inhibitors for non-small cell lung cancer cells.

In some embodiments, the acetyltanshinone IIA is used to prepare an A549 cell growth inhibitor, an H358 cell growth inhibitor, an H1975 cell growth inhibitor, and/or an H1650 cell growth inhibitor, by way of example but not limitation.

In addition, an embodiment of the present disclosure provides the use of acetyltanshinone IIA in the preparation of a protein synthesis inhibitor. In some embodiments, the protein synthesis inhibitor can include an inhibitor of cell cycle-related protein synthesis.

In some embodiments, the protein corresponding to the above-described protein synthesis inhibitor includes, by way of example but not limitation, at least one of p70S6K (p70 ribosomal protein S6 kinase), cyclin D3, AURKA (laser kinase), PLK1 (Polo-like kinase 1), cyclin B1, survivin, EGFR (epidermal growth factor receptor), and MET (hepatocyte growth factor receptor).

On this basis, an embodiment of the present disclosure also provides the use of acetyltanshinone IIA in the preparation of inhibitors for phosphorylation level of signaling molecules downstream of proteins (e.g. for the preparation of p70S6K and/or S6RP phosphorylation inhibitors) and in the preparation of p21 transcriptional activators or p53 promoters.

The present disclosure also provides a medicament for treating lung cancer, the medicament contains acetyltanshinone IIA as a component. In some embodiments, the medicament is an agent for treating non-small cell lung cancer.

In some embodiments, the scope of the present disclosure also includes a lung cancer cell growth inhibitor, a protein synthesis inhibitor, an inhibitor for phosphorylation level of signaling molecules downstream of proteins, a p21 transcriptional activator, and a p53 promoter containing acetyltanshinone IIA in the composition.

In some embodiments, the lung cancer cell growth inhibitor is a growth inhibitor of non-small cell lung cancer cells, such as A549 cell growth inhibitor, H358 cell growth inhibitor, H1975 cell growth inhibitor, and/or H1650 cell growth inhibitor, among others. In some embodiments, the protein synthesis inhibitor is an inhibitor of cell cycle-related protein synthesis, e.g. may be a p70S6K inhibitor, cyclin D3 inhibitor, AURKA inhibitor, PLK1 inhibitor, cyclin B1 inhibitor, survivin inhibitor, EGFR inhibitor, and/or MET inhibitor, etc. In some embodiments, inhibitors for phosphorylation level of signaling molecules downstream of proteins can be an inhibitor of p70S6K and/or S6RP phosphorylation.

The inventors have found that acetyltanshinone IIA (ATA) showed stronger efficacy than Erlotinib in inhibiting the growth of drug-resistant NSCLC cells and their derived xenograft tumors.

ATA achieves these effects primarily through the following mechanisms: first, ATA can bind at the ATP binding site of p70S6K to prevent its phosphorylation, and second, it causes its degradation by increasing ubiquitination of p70S6K. Since p70S6K can induce protein synthesis at the ribosome through phosphorylation of S6 ribosomal protein (S6RP), the dramatic decrease in p70S6K by ATA leads to a dramatic decrease in the synthesis of several cell cycle-related novel proteins, including cyclin D3, aurora kinase A, polo-like kinase, cyclin B1 and survivin; and reduce the levels of EGFR and MET. In addition, ATA increases the levels of p53 and p21 proteins, thereby preventing cell cycle progression in the G1/S phase. Due to the high content of p70S6K in lung tumor samples, ATA degradation of p70S6K can effectively inhibit the growth of TKI-resistant lung cancer cells, so p70S6K may become a new target for treatment of drug-resistant NSCLC cells.

Based on the fact that ATA can effectively block multiple signaling pathways necessary for protein synthesis and cell proliferation, therefore, ATA may be developed as a multi-target anticancer agent for treating TKI drug-resistant NSCLC.

The present disclosure proposes the use of acetyltanshinone IIA for the treatment of lung cancer, especially for the treatment of non-small cell lung cancer, which can antagonize primary and acquired drug resistance of NSCLC cells to epidermal growth factor receptor, a tyrosine kinase inhibitor (EGFR TKIs), by using a small molecule compound acetyltanshinone IIA. The medicament containing acetyltanshinone IIA is expected to become a multi-target anticancer agent for treating TKI drug-resistant NSCLC.

In some embodiments, the pharmaceutical composition further comprises a lung cancer cell growth inhibitor, a protein synthesis inhibitor, an inhibitor for phosphorylation level of signaling molecules downstream of proteins, a p21 transcriptional activator, and a p53 promoter containing acetyltanshinone IIA.

An embodiment of the present disclosure also provides a use of acetyltanshinone IIA for treating a disease associated with lung cancer. An embodiment of the present disclosure also provides the use of a medicament as described above for treating a disease associated with lung cancer.

An embodiment of the present disclosure also provides a method for treating a disease associated with lung cancer in a subject, comprising: administering to a subject in need thereof the medicament as described above.

In some embodiments, the disease associated with lung cancer comprises non-small cell lung cancer and small cell lung cancer. In some embodiments, non-small cell lung cancer comprises adenocarcinoma, squamous cell carcinoma, and large cell carcinoma.

### EXAMPLES

The features and properties of the present disclosure are described in further detail below in connection with examples. The materials and methods involved in the following examples are as follows:

### Cell Lines and Cell Culture

The drug-resistant NSCLC cell lines A549 and H358 were obtained from the American Type Culture Collection (ATCC) and the H1975 and H1650 cell lines from Professor Joong Sup SHIM, college of Health Sciences, University of Macau, China. A549 cells were cultured in Dulbecco's modified Eagle's medium (DMEM) and H358, H1975, and H1650 cells were cultured in RPMI 1640 medium. All media were supplemented with 10% fetal bovine serum (FBS) and 100 U/ml penicillin-streptomycin (both from Gibco). All cells were cultured in a humidified incubator at 37° C. and 5% CO₂ was added.

### Reagents

Available from Selleck Chemicals: Erlotinib (#S7786), Afatinib (#S1011), Osimertinib (#S7297), LY294002 (#S1105), PF-4708671 (#S2163), rapamycin (#S1039) and MLN8237 (#S1133). Available from Sigma-Aldrich: cycloheximide (#01810) and MTT (#M2128) powders.

### MTT Experiment

Cells were seeded at a density of 3×10³ per well in 96-well plates, allowed to attach overnight, and treated with various concentrations of agent. After 72 h of treatment, 10 μL of MTT solution (5 mg/mL) was added to each well and cultured in the plate for 4 h. The crystallized formazan was dissolved in 100 μL of 10% (w/v) SDS and 0.01 mol/L HCl solution for 24 h, and then the value was read by spectrophotometry at 595 nm using a microplate reader. IC₅₀ values refer to the concentration of agent that inhibits cell growth by 50% at 72 h, calculated using GraphPad Prism 7 software.

### Cloning Experiments

Cells were seeded at a density of 1×10³ per well in 6-well plates and cultured for 24 h. The cells were treated with medicament or DMSO for 10 days, after which the cells were washed once with 1×PBS, fixed with 4% paraformaldehyde for 20 min, and stained with 0.1% crystal violet for 15 min. The stained cells in each well were imaged and the number of colonies in each well was quantified using ImageJ software.

### Spheroid Formation Experiment

The density of H1975 was 500 cells per well and the density of H1650 was 1000 cells per well in an ultra-low adhesion round bottom 96 well plate (Corning, #7007). After the cells formed tight spheroids within 48 h, they were exposed to various drug treatments for 8 days with medium changes every 2 days and fresh drug added. Images of spheroids were taken every 2 days (Carl Zeiss Axio Observer 7). The area of the spheroids was calculated using ImageJ software.

### Cell Migration and Invasion

Cell migration and invasion assays were performed in a transwell chamber (Corning, #3422) with a pore size of 8 μm. Cells were treated with different concentrations of ATA and Erlotinib for 48 h, then harvested and resuspended in a serum-free medium. Cells (1×10⁴) were added to the upper side of the transwell chamber and fresh medium containing 10% FBS was added to the lower side. Cells were allowed to migrate from the upper side to the lower side of the chamber at 37° C. for 24 h. Membranes from the cell chamber were fixed with 4% paraformaldehyde for 20 min. The cells remaining on the upper side of the membrane were gently removed with a cotton swab and stained with 1% crystal violet for 20 min to stain the cells migrating to the other side of the transwell chamber. The membrane was then cut and fixed on a glass slide. The migrated cells on each slide were imaged with a Leica M165-FC microscope. For the transwell invasion assay, a Matrigel solution (Corning, #356230) was diluted to one-thirtieth with serum-free medium and applied to the upper side of the precoated chamber for 2 h, after the Matrigel had solidified, the cells were added to the upper side of the transwell chamber. The following procedure is identical to the migration assay. Migrated and invaded cells were quantified using ImageJ software.

### Western Blot Analysis

Cells were washed once with PBS and frozen in RIPA lysis buffer containing protease and phosphatase inhibitors (Sigma-Aldrich) for 30 min. After sonication and centrifugation, the total protein concentration was determined by protein assay using Bio-Rad concentrated dye reagent (Bio-Rad). Equal amounts of protein (25-50 mg) from each sample were separated by SDS-PAGE and electrotransferred to nitrocellulose membranes (GE Healthcare). Detection was performed on the membrane with a 1:1000 dilution of specific primary antibody followed by incubation with a 1:2000 dilution of the HRP conjugated secondary antibody. Finally, immunoreactivity is detected with a chemiluminescent substrate (Clarity Western ECL substrate; Bio-Rad). EGFR (#4267S), MET (#8198S), p-Akt (#9271L), akt (#9272S), p-mTOR (#5536S), mTOR (#2983S), p-p70S6K (#9234S), p70S6K (#2708S), p-S6RP (#4857S), S6RP (#2217S), p-4E-BP1 (#9455S) antibodies. 4E-BP1 (#9644S), p53 (#2527S), p21 waf1/Cip1 (#2947S), survivin (#2808S), cyclin B1 (#12231S), cyclin D3 (#2936S) and GAPDH (#2118S) were purchased from Cell Signaling Technology. Aurora A (#ab13824) and PLK1 (#ab189139) antibodies were from Abcam.

### Immunofluorescence Staining

Cells grown on coverslips were washed once with 1×PBS, fixed with 4% paraformaldehyde for 20 min, and then permeabilized with 0.3% Triton X-100 for 20 min. After blocking with 3% BSA for 1 h, cells were incubated with EGFR (#4267S), MET (#8198S) and p-S6RP (#4857S) antibodies at 1:100 dilution overnight at 4° ° C., and then with Alexa Fluor 488 conjugated anti-rabbit antibodies (Thermo Fisher Scientific) at 1:100 dilution for 1 h. After labeling the nuclei with Hoechst 33342 (Thermo Fisher Scientific) for 15 min, cover slips were mounted onto clean glass microscope slides using Mowiol® 4-88 (Calbiochem, Merck). Photographs of immunofluorescence staining were obtained on a confocal laser scanning microscope (Carl Zeiss Confocal LSM710) equipped with acquisition ZEISS ZEN 2 core imaging software (both from Carl Zeiss Microscopy GmbH).

### Co-Immunoprecipitation (Co-IP)

Cells were disrupted in IP lysis buffer (20 mM Tris-HCl pH 7.6, 150 mM NaCl, 1 mM EDTA, 1% Triton X-100, 5% glycerol) supplemented with protease inhibitors and the deubiquitinase inhibitor N-ethylmaleimide (Sigma-Aldrich) for 30 min on ice. Cell lysates were centrifuged (16,000 g, 4° C., 30 min) and 300 μl supernatant was incubated with 5 μl anti-p70S6K antibody (#sc-8418) overnight at 4° C. The immune complexes were then captured and spun down with 15 ml of Pierce Protein A/G Plus Agarose slurry (Thermo Fisher Scientific) at 4° C. for 4 h. The resin with immunoprecipitates was washed three times, boiled in 2×SDS sample buffer for 5 min, and finally loaded onto an SDS-PAGE gel for Western blot analysis.

### Real-Time PCR

Total RNA was isolated using TRIzol® reagent (Thermo Fisher Scientific) and subsequently reverse-transcribed into first-strand cDNA using the iScript™cDNA synthesis kit (Bio-Rad) according to the manufacturer's instructions. Real-time PCR was performed in triplicate using iTaq™ Universal SYBR Green Supermix (Bio-Rad) on the CFX96 Touch™ Real-Time PCR detection system (Bio-Rad). Relative quantification was performed using the ΔΔCT method. Control samples were used as calibrators to calculate fold changes in the expression of relevant genes in the treated samples. Each real-time quantitative PCR experiment was repeated three times. The primers used for real-time PCR are listed in Table 1.

TABLE 1
Primers for real-time PCR

| | Sequence | | Primer sequence |
|---|---|---|---|
| Gene | number | Direction | (5′ to 3′) |
| AURKA (homo) | SEQ ID NO. 1 | forward | GAGGTCCAAAACGTGTTCTCG |
| | SEQ ID NO. 2 | reverse | ACAGGATGAGGTACACTGGTTG |
| BIRC5 (homo) | SEQ ID NO. 3 | forward | AGGACCACCGCATCTCTACAT |
| | SEQ ID NO. 4 | reverse | AAGTCTGGCTCGTTCTCAGTG |
| CCNB1 (homo) | SEQ ID NO. 5 | forward | AACTTTCGCCTGAGCCTATTTT |
| | SEQ ID NO. 6 | reverse | TTGGTCTGACTGCTTGCTCTT |
| CCND3 (homo) | SEQ ID NO. 7 | forward | TACCCGCCATCCATGATCG |
| | SEQ ID NO. 8 | reverse | AGGCAGTCCACTTCAGTGC |
| CDKNIA (homo) | SEQ ID NO. 9 | forward | CGATGGAACTTCGACTTTGTCA |
| | SEQ ID NO. 10 | reverse | GCACAAGGGTACAAGACAGTG |
| EGFR (homo) | SEQ ID NO. 11 | forward | AGGCACGAGTAACAAGCTCAC |
| | SEQ ID NO. 12 | reverse | ATGAGGACATAACCAGCCACC |
| GAPDH (homo) | SEQ ID NO. 13 | forward | GTCAGTGGTGGACCTGACCT |
| | SEQ ID NO. 14 | reverse | AAAGGTGGAGGAGTGGGTGT |
| MET (homo) | SEQ ID NO. 15 | forward | AGCAATGGGGAGTGTAAAGAGG |
| | SEQ ID NO. 16 | reverse | CCCAGTCTTGTACTCAGCAAC |
| PLK1 (homo) | SEQ ID NO. 17 | forward | AAAGAGATCCCGGAGGTCCTA |
| | SEQ ID NO. 18 | reverse | GGCTGCGGTGAATGGATATTTC |
| RPS6KB1 (homo) | SEQ ID NO. 19 | forward | AGAACTTCTGGCTCGAAAGGT |
| | SEQ ID NO. 20 | reverse | CGACAGGTGTCTGACGTGTAA |

### RNA Sequencing Analysis

Total RNA was extracted from A549 cells treated with ATA or control for 48 h using TRIzol® reagent (Thermo Fisher Scientific). The RNA samples were then submitted to Novogene (Beijing, China) for IlluminaHiseq PE150 sequencing.

### Molecular Docking Analysis

Molecular docking analyses were performed to investigate the binding of ATA and HTA to p70S6K (PDB ID: 4RLO), all obtained from protein databases. All docking uses AutoDock Vina software.

### Liquid Chromatography-Mass Spectrometry (LC-MS) Analysis

Cells were treated with 2 μM ATA for 0-6 h, after extensive washing, cells were harvested and lysed following the co-IP protocol with p70S6K antibody. The co-IP pulled immunoprecipitate was treated with two volumes of cold acetonitrile containing 1 mM DTT. Analysis was performed by LC-MS (Waters Xevo TQD) under the following conditions. The A-line mobile phase was increasing concentrations of acetonitrile (0 min 60%, 1 min to 4 min 60%, 5 min 90%) and the B-line mobile phase was 0.1% formic acid at a flow rate of 0.4 mL/min. Acetonitrile was used as a negative control and a 0.1 μM ATA standard was used as a positive control.

### Cell Cycle Analysis

Cell cycle analysis was performed using standard flow cytometer protocols. Briefly, after ATA treatment, cells were harvested, washed with 1×PBS, and then fixed with 70% pre-chilled ethanol at 4° C. for 30 min. Fixed cells were washed twice with 1×PBS, resuspended in 0.5 mL of 1×PBS containing 50 μg/mL propidium iodide (PI) and 100 μg/mL RNase A (ribonuclease A) for 30 min at 37° C. in the dark. The cell cycle was then analyzed on a BD Accuri C6 flow cytometer (BD Biosciences, CA). Data from the flow cytometer were analyzed using FlowJo software (Tree Star).

### siRNA-Based Gene Knockout

Lipofectamine 2000 transfection reagent (#11668019) was purchased from Invitrogen (Waltham, USA) and AllStars negative control (#1027280) siRNA was purchased from Qiagen (Hilden, Germany). Sip70S6K-1 (5′ to 3′ sequence: CAUGGAACAUUGAGAAA) (SEQ ID NO. 21) and Sip70S6K-2 (5′ to 3′ sequence: GGUUUUCAAGUACGAAAA) (SEQ ID NO. 22) siRNA was designed by us. siRNA transfection experiments were performed according to the manufacturer's instructions. Briefly, 3 μL of siRNA at a stock concentration of 10 μM was suspended in 250 μL of serum-free medium and mixed with 250 μL of serum-free medium containing 6 μL Lipofectamine 2000. The transfection mixture was incubated at room temperature for 20 min. The cells were trypsinized and 3×10⁵ cells were suspended in 2.5 mL of culture medium and added to a 60 mm culture dish. The transfection mixture was added dropwise to the suspended cells in the culture dish. Western blot analysis with anti-p70S6K antibody was performed to assess the efficiency of gene silencing.

### Gene Overexpression

The RPS6KB1 plasmid (pLV[Exp]-Puro-CMV>hRPS6KB1) and the AURKA plasmid (pLV[Exp]-Puro-CMV>hAURKA) were purchased from VectorBuilder. Plasmids were transfected into host cells using a lentivirus-based infection method. Briefly, 239T cells were seeded in 6-well plates at a density of 8×10⁵. After 12-16 h, cells were transfected with pMD2G (encoding the VSV G envelope protein), pCMVR8.2 (encoding the HIV-1 Gag, Pol, Tat, and Rev proteins) and the plasmids of interest (pCDH, pRPS6KB1 and pAURKA). After 4-6 h of transfection, the transfection solution was replaced with a fresh complete medium. The virus-containing supernatant was collected after 36 h. Lung cancer H1650 and A549 cells were seeded in 6-well plates at a density of 2×10⁵ for 24 h, and the virus produced after infection with 239T cells was incubated with lung cancer cells for 24 h and then replaced with fresh medium. Cells were screened with 2 μg/ml puromycin for several days.

### Immunohistochemistry

Tumor xenograft tissues formed in nude mice were fixed in 10% neutral buffered formalin overnight at room temperature, processed into paraffin blocks, and then sectioned at a thickness of 5 μm. The tissue sections and the patient's tumor tissue were deparaffinized and then boiled with citrate buffer for 5 min to expose the antigen. After blocking endogenous peroxidase activity and non-specific antibody binding, sections were incubated with the primary antibody overnight at 4° C. Immunoreactivity was detected using the rabbit-specific HRP/DAB (ABC) detection IHC kit (Abcam) according to the manufacturer's instructions. Sections were lightly counterstained with hematoxylin. Color images of immunohistochemical staining were obtained on a light microscope using a Zeiss Axiocam 506 color camera (Carl Zeiss Microscopy GmbH).

### Mouse Xenograft Study

Cell line xenograft experiments were performed in 6-week-old female nude mice, 4×10⁶ A549 cells were mixed 1:1 with matrigel-basement membrane matrix (Corning) and injected subcutaneously into nude mice. Tumors were allowed to grow to around 80 mm and mice were randomly selected to receive treatment with ATA (25 mg/kg) or Erlotinib (25 mg/kg) intraperitoneally every 3 days for 31 days. ATA was formulated with 25% ethanol, 60% PEG300 (Sigma-Aldrich), and 15% Tween 80 (Sigma-Aldrich). Erlotinib was formulated in 5% DMSO, 30% PEG300, 5% Tween 80 and 60% H₂O. Mouse body weight and tumor volume were measured every 3 days by a scale and caliper. Tumor volume (mm³) was calculated using the formula. π/6×length (mm)×[width (mm)]². At least 6 mice in the control and treatment groups were evaluated during the study. All mice were sacrificed after 31 days of drug treatment, tumors were collected, and tumor weights were recorded. All animal studies were conducted in accordance with the requirements of the Animal Research Ethics Committee of Macao University and in compliance with all relevant ethical requirements.

### Statistical Analysis

All experiments were performed in triplicate. Data are expressed as mean±standard deviation (SD). The statistical significance of the control and test groups was determined by the relevant test of GraphPad Prism 7. The significance is shown below. Based on one-way or two-way analysis of variance followed by Tukey's multiple comparison test or Student's t-test, *P<0.05, **P<0.01, ***P<0.001, and ****P<0.0001.

### Example 1

### ATA Effectively Inhibits the Growth of NSCLC Cells That Produce Primary or Acquired Drug Resistance to EGFR TKIs

Four drug-resistant cell lines were selected, of which A549 and H358 cells had primary resistance to EGFR TKIs (e.g. Erlotinib, Afatinib, and Osimertinib) due to activating mutations in wild-type EGFR (wt-EGFR) and KRAS. H1975 cells have two mutations in EGFR (L858R and T790M) and the second mutation makes these cells resistant to Erlotinib. H1650 cells have one EGFR activating mutation (A746-A750 deletion) and one PTEN deletion mutation. Due to the deletion of PTEN, these cells developed resistance to three anti-EGFR drugs, Erlotinib, Afatinib, and Osimertinib (as shown in Table 2).

TABLE 2
IC₅₀ values of Erlotinib and ATA in drug-resistant NSCLC cells

| | EGFR status | K-RAS | PTEN | Drug | IC₅₀ (μM) | | IC₅₀ ratio of |
|---|---|---|---|---|---|---|---|
| Cell line | (status) | status | status | resistance | Erlotinib | ATA | Erlotinib/ATA |
| A549 | WT | G12S | WT | Primary | 19.59 ± 0.28 | 1.83 ± 0.21 | 10.7 |
| H358 | WT | G12C | WT | Primary | 21.48 ± 1.47 | 1.57 ± 0.10 | 13.7 |
| H1975 | L858R/T790M | WT | WT | Acquired | 10.55 ± 1.27 | 1.76 ± 0.02 | 6 |
| H1650 | Deletion | WT | PTEN | Acquired | 22.08 ± 0.59 | 1.28 ± 0.29 | 17.3 |
| | (E746-A750) | | deletion | | | | |

WT: wild type

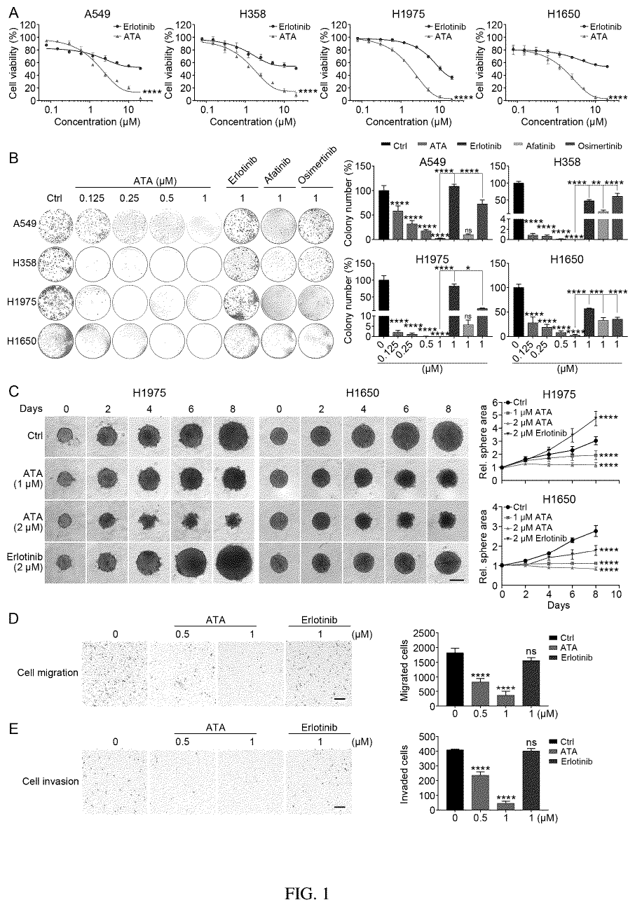

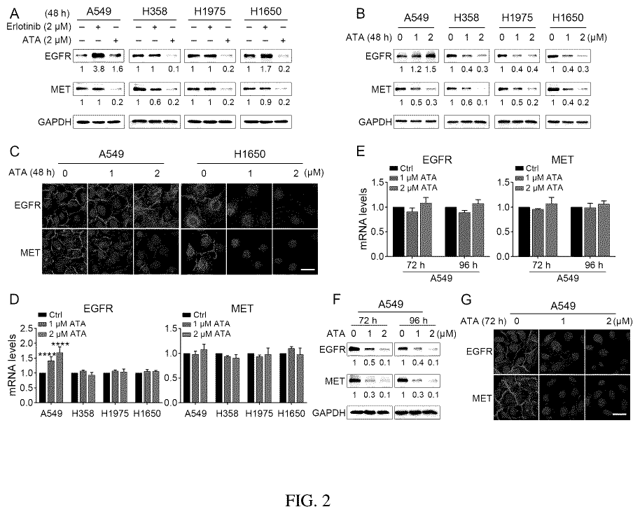

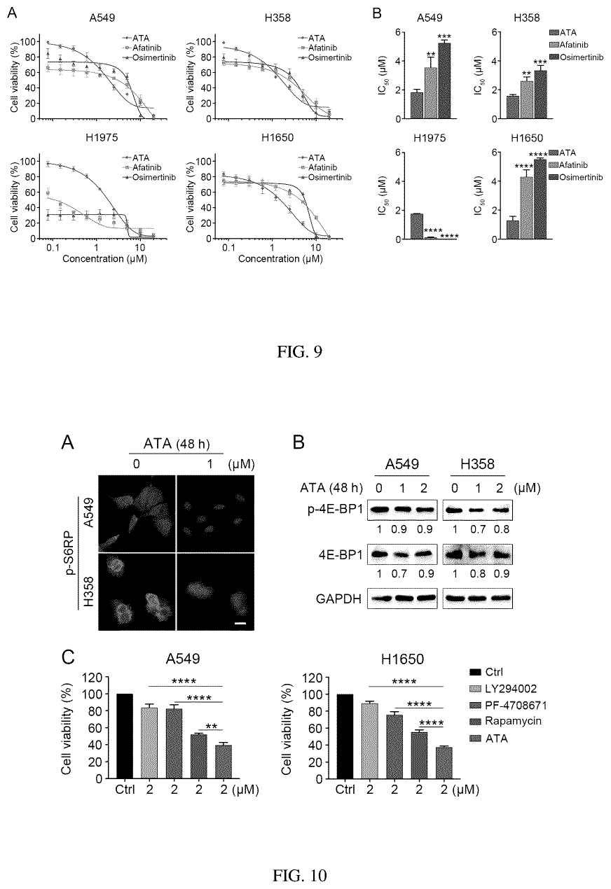

ATA was compared to first-generation EGFR TKI in three EGFR TKIs: growth inhibition by Erlotinib, MTT results showed that in all four cell lines, ATA treatment significantly reduced cell viability compared to Erlotinib (as shown in A in FIG. 1). The IC₅₀ values of Erlotinib in these cells ranged from 10-22 μM, whereas the IC₅₀ values of ATA ranged from 1.3-1.8 μM, 6-17 fold lower than that of Erlotinib (Table 2). Subsequently, the viability of all four cell lines was measured after treatment of ATA, second-generation EGFR TKI: Afatinib, or third-generation EGFR TKI: Osimertinib treatment (as shown in A in FIG. 9), and corresponding IC₅₀ values were calculated. The IC₅₀ values of ATA were significantly lower than Afatinib and Osimertinib for A549, H358, and H1650 cells, while the IC₅₀ values of ATA were significantly higher than these two inhibitors for H1975 cells (as shown in B in FIG. 9).

Subsequently, the inhibition of colony formation by ATA was compared with three inhibitors. Results show that at 1 μM, ATA inhibited colony formation of all four drug-resistant cell lines much more effectively than Erlotinib and Osimertinib. In H358 and H1650 cells, ATA also showed significantly higher colony formation inhibition than Afatinib. Importantly, even at low concentrations of 0.125 μM, ATA almost completely inhibited colony formation of H358 and H1975 cells and significantly reduced colony colony-forming capacity of H1650 and A549 cells by 73% and 42%, respectively (as shown in B in FIG. 1).

In addition, spheroid formation assays were used to evaluate the ability of ATA to inhibit the growth of two NSCLC cell lines with acquired drug resistance under three-dimensional and non-adherent conditions. Results show that ATA strongly inhibited spheroid formation in H1975 and H1650 cells at both 1 and 2 μM concentrations; spheroid growth of both cell lines was completely inhibited at 2 μM.

In contrast, Erlotinib at 2 μM did not decrease but significantly increased the sphere size of H1975 cells, producing less inhibition in H1650 cells than ATA (as shown in C in FIG. 1).

Finally, in order to assess the anti-metastatic potential of ATA in drug-resistant NSCLC cells, cell migration and invasion assays were performed. Results show that ATA at 1 μM significantly reduced the migration and invasion ability of A549 cells by 80% and 88%, respectively. Even at 0.5 μM, ATA produced a significant inhibitory effect on cell migration and invasion, whereas Erlotinib did not (as shown in D and E in FIG. 1).

Accordingly, the results of the four in vitro experiments show that ATA can effectively inhibit the growth, migration, and invasion of NSCLC cells with primary or acquired drug resistance to EGFR TKIs.

In FIG. 1, (A) is the cell viability determined by the MTT method for A549, H358, H1975, and H1650 cells treated with different concentrations of ATA or Erlotinib for 72 h. ****P<0.0001 was based on a two-way analysis of variance followed by Sidak's multiple comparison test. (B) is A549, H358, H1975, and H1650 cells treated with different concentrations of ATA, Erlotinib (1 μM), Afatinib (1 μM) or Osimertinib (1 μM) for 10 days. The plates were stained with crystal violet. Representative images of three independent experiments are shown. The colonies formed were quantified (relative number of colonies compared to the control group). *P<0.05, **P<0.01, ***P<0.001, and ****P<0.0001 were based on one-way analysis of variance and Tukey's multiple comparison tests. (C) are H1975 and H1650 cell spheres treated with ATA (1 or 2 μM) or Erlotinib (2 μM) for 0 to 8 days, respectively. Representative images of three independent experiments are shown. The quantification of the relative sphere area is the average of 8 spheres (N=8). ****P<0.0001 was based on a two-way analysis of variance and Tukey's multiple comparison test. Scale bar, 200 μm. (D) is A549 cells treated with ATA (0.5 or 1 μM) or Erlotinib (1 μM) for 24 h and migrated cells were stained with crystal violet. Quantitative display of migrated cells. ****P<0.0001 was based on a one-way analysis of variance and Tukey's multiple comparison test. Scale bar, 200 μm. (E) are A549 cells treated with ATA (0.5 or 1 μM) or Erlotinib (1 μM) for 24 h and the invading cells were stained with crystal violet. Quantification of invaded cells. ****P<0.0001 was based on a one-way analysis of variance followed by Tukey's multiple comparison test. Scale bar, 200 μm. Data are expressed as the mean±SD of three independent experiments.

In FIG. 9, (A) is cell viability determined using the MTT assay A549, H358, H1975, and H1650 cells were treated with different concentrations of ATA, Afatinib, or Osimertinib for 72 h. (B) is IC₅₀ values for ATA, Afatinib and Osimertinib in A549, H358, H1975 and H1650 cells. ****P<0.01, ***P<0.001 and ****P<0.0001 were based on two-tailed analysis and t-test. Data are expressed as the mean±SD of three independent experiments.

### Example 2

### ATA is More Effective Than Erlotinib in Reducing Protein Levels of EGFR and MET in Drug-Resistant NSCLC Cells

To determine why ATA was more effective than Erlotinib in inhibiting the growth of drug-resistant NSCLC cells, this example compared their effects on EGFR and MET protein levels in all cell lines used in this study. Results show that Erlotinib treatment significantly increased the protein levels of EGFR in A549 and H1650. However, in addition to the EGFR protein levels of A549, ATA strongly reduced the EGFR and MET protein levels of drug-resistant NSCLC cells (as shown in A in FIG. 2).

Further, to confirm whether ATA can inhibit the growth of drug-resistant NSCLC by reducing the levels of EGFR and MET, A549, H358, H1975 and H1650 cells were treated with 1 or 2 μM of ATA for 48 h, and the results of Western blot showed that EGFR protein levels in H358, H1975, and H1650 cells were greatly reduced by 60-70% by 1 or 2 μM ATA, and MET protein levels in all four cell lines were reduced by 70-90% by 2 μM ATA (as shown in B in FIG. 2). Immunofluorescence staining images further confirmed: ATA effectively reduced the levels of EGFR protein in H1650 cells, and reduced the levels of MET protein in A549 and H1650 cells (as shown in C in FIG. 2).

To explain that 48 h ATA treatment did not decrease but increased EGFR protein levels in A549 cells, mRNA levels were determined after ATA treatment. Real-time PCR results show that after 48 h of ATA treatment, mRNA levels of EGFR in A549 cells were significantly increased, but mRNA levels of EGFR or MET in the other three cell lines H358, H1975, and H1650 were not affected (as shown in D in FIG. 2). This transient increase in EGFR mRNA levels may result in increased protein levels following ATA treatment in A549 cells. To test this hypothesis, extending the treatment time of ATA from 48 h to 72 and 96 h, it was found that ATA did not significantly increase the mRNA levels of EGFR or MET in A549 cells over these longer times (as shown in E in FIG. 2). Also, treatment of A549 cells with 2 μM ATA for 72 or 96 h decreased protein levels of EGFR and MET in the cells by as much as 90% (as shown in F and G in FIG. 2).

The results show that ATA can effectively reduce the protein levels of EGFR and MET in NSCLC cells resistant to primary or acquired EGFR TKIs.

In FIG. 2 (A) is protein levels of EGFR and MET determined by Western blot analysis for A549 and H1650 cells treated with or without Erlotinib (2 μM) or ATA (2 μM) for 48 h. (B) is protein levels of EGFR and MET determined by Western blot analysis for A549, H358, H1975, and H1650 cells treated with ATA (1 or 2 μM) for 48 h. (C) is representative confocal images showing EGFR and MET immunofluorescence staining for A549 and H1650 cells treated with ATA (1 or 2 μM) for 48 h. Scale bar, 20 μm. (D) are EGFR and MET mRNA levels measured by real-time PCR for A549, H358, H1975, and H1650 cells treated with ATA (1 or 2 μM) for 48 h. Quantification of EGFR and MET mRNA levels. ****P<0.0001 was based on a two-way analysis of variance followed by Tukey's multiple comparison test. (E) is EGFR and MET mRNA levels measured by real-time PCR for A549 cells treated with ATA (1 or 2 μM) for 72 and 96 h. Quantification of EGFR and MET mRNA levels is shown. (F) are EGFR and MET protein levels determined by Western blot analysis for A549 cells treated with ATA (1 or 2 μM) for 72 and 96 h. (G) is representative confocal images showing EGFR and MET immunofluorescence staining for A549 cells treated with ATA (1 or 2 μM) for 72 h. Scale bar, 20 μm. Data are expressed as the mean±SD of three independent experiments.

### Example 3

### ATA Inhibits the Growth of Drug-Resistant NSCLC Cells by Reducing p70S6K

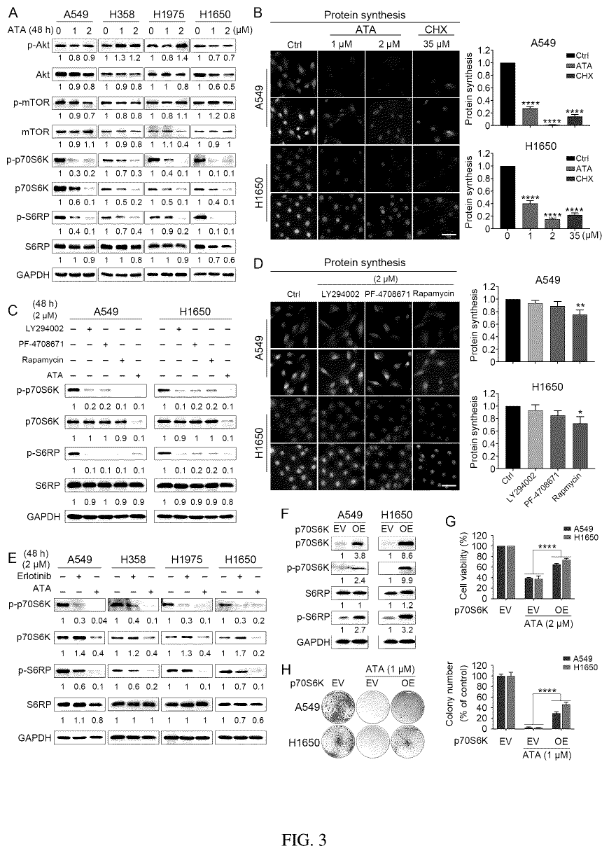

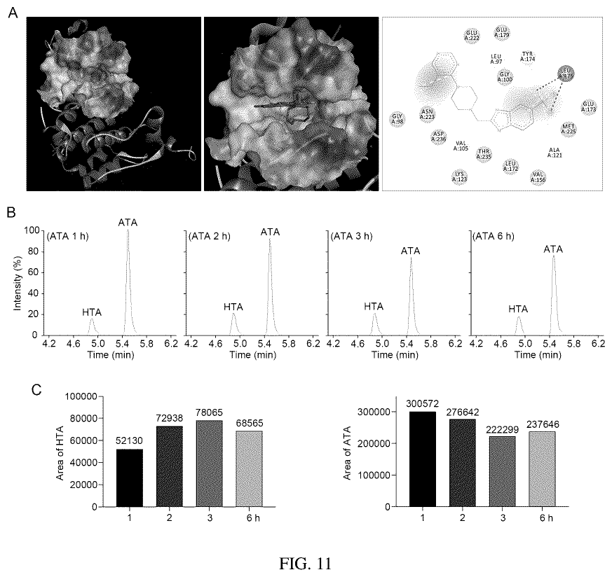

The effect of ATA on the downstream signaling pathways of EGFR and MET was investigated and Western blot results showed that ATA treatment did not significantly reduce the levels of p-Akt, Akt, p-mTOR, and mTOR in most cell lines, but greatly reduced the levels of p70S6K protein and its phosphorylated forms. In addition, ATA also significantly reduced the phosphorylation of S6 ribosomal protein (S6RP) which can be phosphorylated by p70S6K; however, ATA did not significantly reduce the level of S6RP protein (as shown in A in FIG. 3). Confocal immunofluorescence staining also demonstrated that ATA treatment reduced the levels of p-S6RP in A549 and H358 cells (as shown in A in FIG. 10).

Since phosphorylation of S6RP by p70S6K induces protein synthesis by ribosomes, experiments were performed to determine whether the reduction of p70S6K by ATA affects the synthesis of new proteins, the results of which show that ATA significantly inhibited the synthesis of new proteins in A549 and H1650 cells. In particular, 2 μM ATA almost completely abolished the synthesis of a new protein and achieved a better inhibitory effect than cyclohexylamine (CHX, a protein synthesis inhibitor) (as shown in B in FIG. 3). In addition to p70S6K, the effect of ATA on eukaryotic promoter 4E-binding protein 1 (4E-BP1) was investigated. In contrast to its effect on p70S6K, ATA had little effect on reducing the levels of total and phosphorylated 4E-BP1 (as shown in B in FIG. 10). These results show that ATA may inhibit protein synthesis by decreasing protein levels of p70S6K and inhibiting phosphorylation of p70S6K and S6RP.

To determine whether ATA inhibits the growth of drug-resistant NSCLC cells by reducing the protein level of p70S6K or inhibiting its phosphorylation, the effects of ATA in reducing the levels of p70S6K and its phosphorylated form in A549 and H1650 cells were compared to the effects of PI3K (LY294002), p70S6K (PF-4708671) and mTOR (rapamycin) inhibitors. Western blot results showed that ATA significantly decreased the levels of p70S6K, p-p70S6K, and p-S6RP; in contrast, the other three inhibitors did not decrease the levels of p70S6K but only decreased the levels of p-p70S6K and p-S6RP (as shown in C in FIG. 3). Further, the efficacy of inhibiting protein synthesis was compared between ATA and the three inhibitors. Results show that LY294002 and PF-4708671 did not affect protein synthesis at 2 μM. Although Rapamycin reduced protein synthesis by 25-28% in A549 and H1650 cells (as shown in D in FIG. 3), ATA achieved a higher inhibition ratio of 85-99% at the same concentration (as shown in B in FIG. 3). These results indicate that ATA prevents protein synthesis more effectively than inhibitors of PI3K, p70S6K, and mTOR. Further, the growth inhibitory effects of ATA and these three inhibitors were compared in A549 and H1650 cells. MTT results show that the inhibitory effect of ATA on the growth of A549 and H1650 cells was significantly higher than all three inhibitors (as shown in C in FIG. 10).

In addition, drug-resistant NSCLC cells showed higher p70S6K levels after Erlotinib treatment (as shown in E in FIG. 3), which may result in resistance of these cells to Erlotinib. To confirm that ATA inhibits the growth of drug-resistant NSCLC cells by decreasing p70S6K, p70S6K protein was overexpressed in A549 and H1650 cells, and its overexpression was observed to increase the levels of p70S6K, p-p70S6K, p-S6RP, but not affect the level of S6RP protein (as shown in F in FIG. 3). Subsequent treatment of these cells with ATA revealed that overexpression of p70S6K in these cells partially reversed the growth inhibition effect of ATA in two-dimensional culture (as shown in G in FIG. 3). Furthermore, overexpression of p70S6K in A549 and H1650 cells significantly reversed the inhibitory effect of ATA on colony formation (as shown in H in FIG. 3).

Accordingly, the results show that ATA has two effects on p70S6K: while inhibiting its phosphorylation and reducing its protein levels, ATA can inhibit the growth of drug-resistant NSCLC cells by decreasing p70S6K and inhibiting protein synthesis. In FIG. 3 (A) is protein levels of p-Akt, Akt, p-mTOR, mTOR, p-p70S6K, p70S6K, p-S6RP, and S6RP determined by Western blot analysis for A549, H358, H1975, and H1650 cells treated with ATA (1 or 2 μM) for 48 h. (B) is A549 and H1650 cells treated with ATA (1 or 2 μM) or the protein synthesis inhibitor cyclohexylamine (CHX, 35 μM), respectively, for 48 h. The rate of protein synthesis was determined using the click-iT™ HPG Alexa Fluor™ 594 Protein Synthesis Assay Kit. The intensity of Texas Red fluorescence in the stained image represents the rate of protein synthesis. Quantification of the rate of protein synthesis is shown. ****P<0.0001 was based on a one-way analysis of variance followed by Tukey's multiple comparison test. Scale bar, 50 μm. (C) is A549 and H1650 cells treated with PI3K inhibitor (LY294002, 2 μM), p70S6K inhibitor (PF-4708671, 2 μM), mTOR inhibitor (Rapamycin, 2 μM) or ATA (2 μM), respectively, for 48 h. Protein levels of p-p70S6K. p70S6K. p-S6RP, and S6RP were determined by Western blot analysis. (D) is A549 and H1650 cells treated with PI3K inhibitor (LY294002, 2 μM), p70S6K inhibitor (PF-4708671, 2 μM), or mTOR inhibitor (Rapamycin, 2 μM), respectively, for 48 h. The rate of protein synthesis was determined using the click-iT™ HPG Alexa Fluor™ 594 Protein Synthesis Assay Kit. The intensity of Texas Red fluorescence in the stained image represents the rate of protein synthesis. Quantification of the rate of protein synthesis is shown. *P<0.05 and **P<0.01 were based on a one-way analysis of variance followed by Tukey's multiple comparison test. Scale bar, 50 μm. (E) Protein levels of p-p70S6K. p70S6K. p-S6RP, and S6RP were determined by Western blot analysis for A549, H358, H1975, and H1650 cells treated with or without Erlotinib (2 μM) or ATA (2 μM) for 48 h. (F) is transfecting A549 and H1650 cells with empty vector (EV) or p70S6K overexpression (p70S6K-OE) plasmids. The empty vector (EV) plasmid was used as a control. Protein levels of p70S6K. p-p70S6K, S6RP, and p-S6RP were determined by Western blot analysis. (G) is the cell viability determined by the MTT method for A549 and H1650 cells overexpressing p70S6K treated with ATA (2 μM) for 72 h. ****P<0.0001 was based on a one-way analysis of variance and Tukey's multiple comparison test. (H) is A549 and H1650 cells overexpressing p70S6K treated with ATA (1 μM) for 10 days. The plates were stained with crystal violet. Representative images of three independent experiments are shown. Quantification of colony formation (relative number of colonies compared to that of the control group) is shown. ****P<0.0001 was based on a one-way analysis of variance followed by Tukey's multiple comparison test. Data are expressed as the mean±SD of three independent experiments.

In FIG. 10, (A) is A549 and H358 cells treated with ATA (1 μM) for 48 h. Representative confocal images of p-S6RP immunofluorescence staining are shown. Scale bar, 20 μm. (B) is A549 and H358 cells treated with ATA (1 or 2 μM) for 48 h. Protein levels of p-4E-BP1 and 4E-BP1 were determined by Western blot analysis. Data are expressed as the mean±SD of three independent experiments. (C) is A549 and H1650 cells treated with PI3K inhibitor (LY294002, 2 μM), p70S6K inhibitor (PF-4708671, 2 μM), mTOR inhibitor (Rapamycin, 2 μM) or ATA (2 μM), respectively, for 72 h. Cell viability was then determined by the MTT method. **P<0.01 and ****P<0.0001 were based on a one-way analysis of variance followed by Tukey's multiple comparison test.

### Example 4

ATA Degrades p70S6K Protein by Binding to p70S6K and Increasing the Ubiquitination Thereof

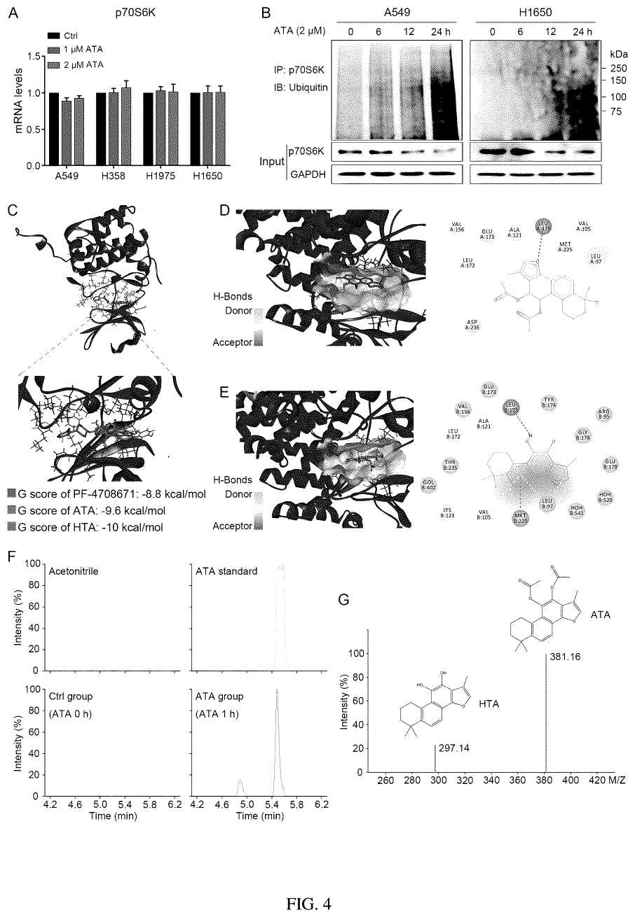

To investigate how ATA reduces p70S6K protein levels, p70S6K mRNA levels were first measured and qPCR results showed that ATA did not alter mRNA levels of p70S6K in all four cell lines (as described for A in FIG. 4). Subsequent isolation of p70S6K from cell lysates with Co-IP, ATA treatment was found to greatly increase ubiquitination of p70S6K in A549 and H1650 cells (as shown in B in FIG. 4). To investigate whether ATA might induce protein degradation by binding to p70S6K, molecular docking assays were performed, in particular, using the chemical structures of ATA and HTA. The chemical structure of PF-4708671 was used as a positive control to locate the ATP binding pocket in p70S6K in a docking assay. Results show that PF-4708671 can bind to the ATP binding pocket of p70S6K and form a hydrogen bond with p70S6K Leu-175 amino acid residues (as shown in A in FIG. 11). Molecular docking analysis was subsequently performed with p70S6K using ATA and HTA. It was found that ATA and HTA can bind to p70S6K at the same position where PF-4708671 binds to p70S6K, i.e. the ATP binding pocket (as shown in C in FIG. 4). The interactions in the model show that both ATA and HTA interact with amino acid residues in the ATP binding pocket and form hydrogen bonds with p70S6K through Leu-175 amino acid residues (as shown in D and E in FIG. 4), which is consistent with the binding site of PF-4708671 in p70S6K (as shown in A in FIG. 11). In addition, the binding energies between ATA, HTA and PF-4708671 and p70S6K were calculated respectively and the results showed that HTA has the lowest binding energy of −10.0 kcal/mol, slightly lower than ATA (−9.6 kcal/mol), but much lower binding energy (G score) (−8.8 kcal/mol) than the known inhibitor (PF-4708671) (as shown in C in FIG. 4). These results show that ATA and HTA may bind to p70S6K more efficiently than PF-4708671.

To further investigate whether ATA and HTA can bind to p70S6K, p70S6K was isolated from cell lysates using co-IP, followed by the detection of ATA and HTA by LC-MS. The elution profile of the LC-MS showed that no distinct peak was detected in the acetonitrile group (negative control), but one peak was detected in the ATA standard (positive control) (as shown in F in FIG. 4). Subsequently, the control group (cells treated with ATA for 0 h) and the ATA group (cells treated with ATA for 1 h) were tested with the same conditions. Results show that no distinct peak was detected in the control group, while two peaks were detected in the ATA group (as shown in F in FIG. 4). Subsequently, both peaks were analyzed by mass spectrometry. The first small peak had m/z=297.14, which is likely from HTA (MW=296.14) plus an H⁺ (MW=1.00). The second largest peak had m/z=381.16, likely from ATA (MW=380.16) and one H⁺ (MW=1.00) (as shown in G in FIG. 4). These results show that both ATA and HTA bind to p70S6K.

In addition, LC-MS was used to detect ATA and HTA in the ATA group at different periods (1, 2, 3, and 6 h) and the quantification results showed that the HTA content of p70S6K binding increased within 1-3 h and decreased slightly within 6 h, while the ATA content of p70S6K binding decreased progressively within 1-3 h and remained relatively stable after 6 h (as shown in B and C in FIG. 11). These are likely due to ATA binding to p70S6K primarily in the first hour. When more ATA molecules are converted to HTA, HTA can also bind to p70S6K, increasing its binding to p70S6K by 50% within 3 h.

Accordingly, the above results show that ATA can rapidly bind to p70S6K, which may prevent its phosphorylation and stimulate ubiquitination-mediated protein degradation.

In FIG. 4, (A) is p70S6K mRNA levels measured by real-time PCR for A549, H358, H1975, and H1650 cells treated with ATA (1 or 2 μM) for 48 h. Quantitative display of p70S6K mRNA levels. (B) is ubiquitin and p70S6K protein levels determined by Western blot analysis, after 6, 12, and 24 h of treatment of A549 and H1650 cells with ATA (2 μM) followed by p70S6K and the ubiquitination products isolated from cell lysates with anti-p70S6K antibody. (C) are schematic diagrams showing the binding of ATA, HTA, and the p70S6K inhibitor PF-4708671 to the ATP binding pocket of the p70S6K protein. (D) and (E) are schematic diagrams showing the binding of ATA and HTA to the ATP binding pocket of the p70S6K protein. The model interaction plot shows that ATA and HTA bind to amino acid residues in the p70S6K protein. The purple dashed lines indicate electrostatic interactions and the green dashed lines indicate hydrogen bonding. (F) is the LC-MS chromatograms of acetonitrile, ATA standard, control, and ATA groups. (G) is the mass spectrometry used to determine the molecular weights of ATA and HTA. Data in A shows the mean±SD of three independent experiments.

In FIG. 11, (A) are schematic diagrams showing the ATP binding pocket of the p70S6K protein. The magnified image shows that the p70S6K inhibitor (PF-4708671) binds to the ATP binding pocket of the p70S6K protein. The model interaction plot shows that PF-4708671 binds to amino acid residues in the p70S6K protein. The keys are as shown in dashed lines and are color-coded as follows: purple is an electrostatic interaction and green is a hydrogen bond. (B) are LC-MS chromatograms of ATA-treated groups at 1, 2, 3, and 6 h. (C) are quantification results of HTA and ATA curve areas of the ATA treatment group at 1, 2, 3, and 6 h.

### Example 5

### ATA Prevents Cell Cycle Progression in the G1/S Phase by Affecting p21 and Cyclin D3

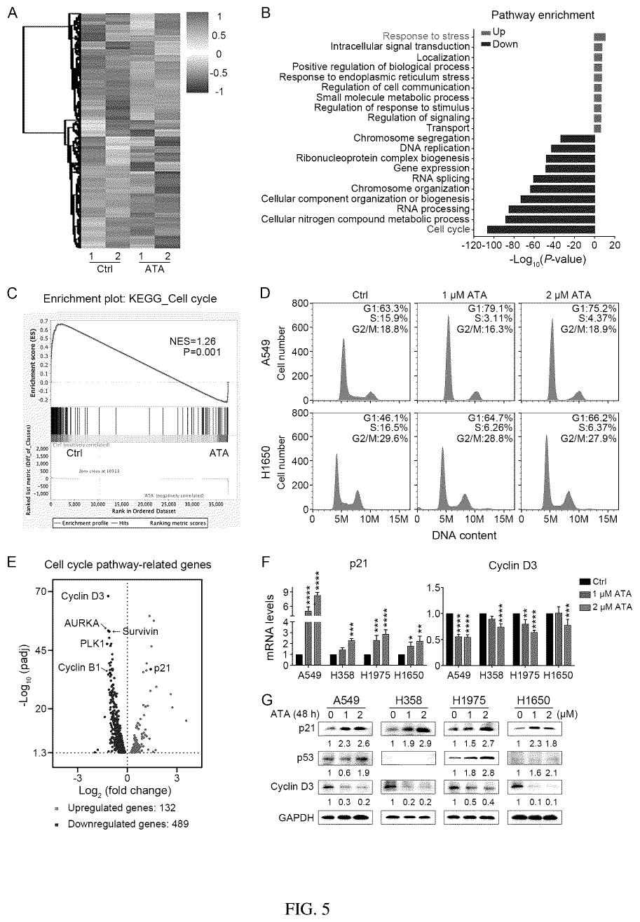

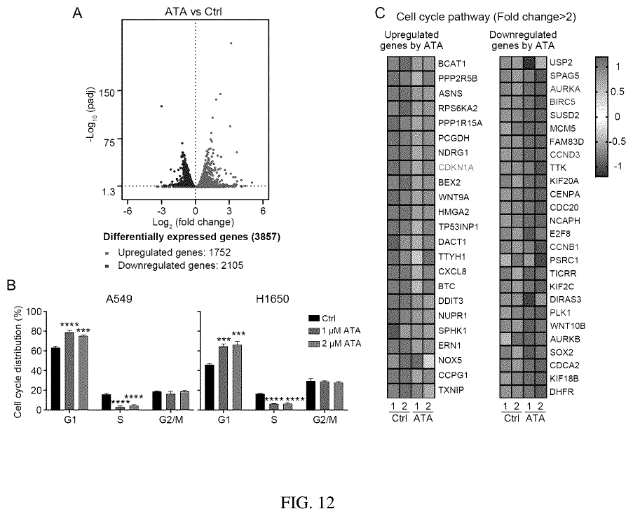

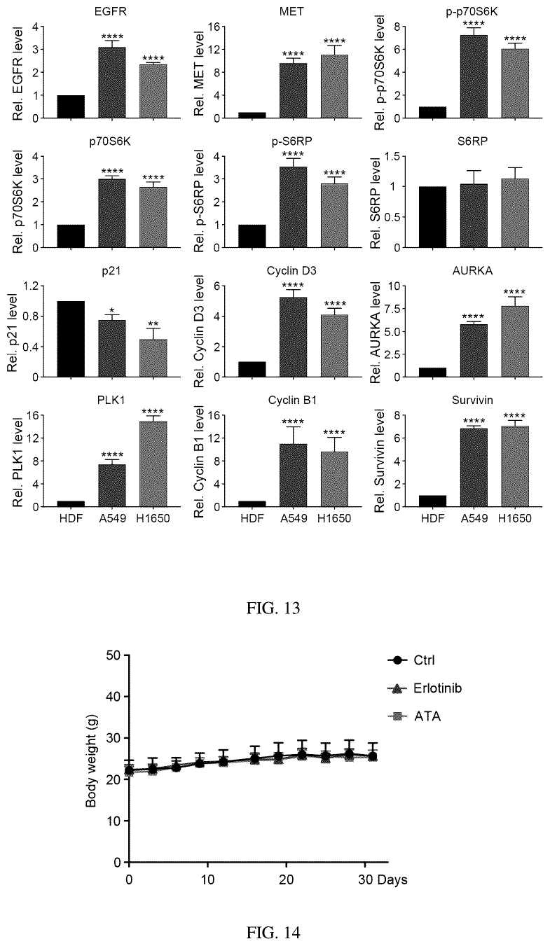

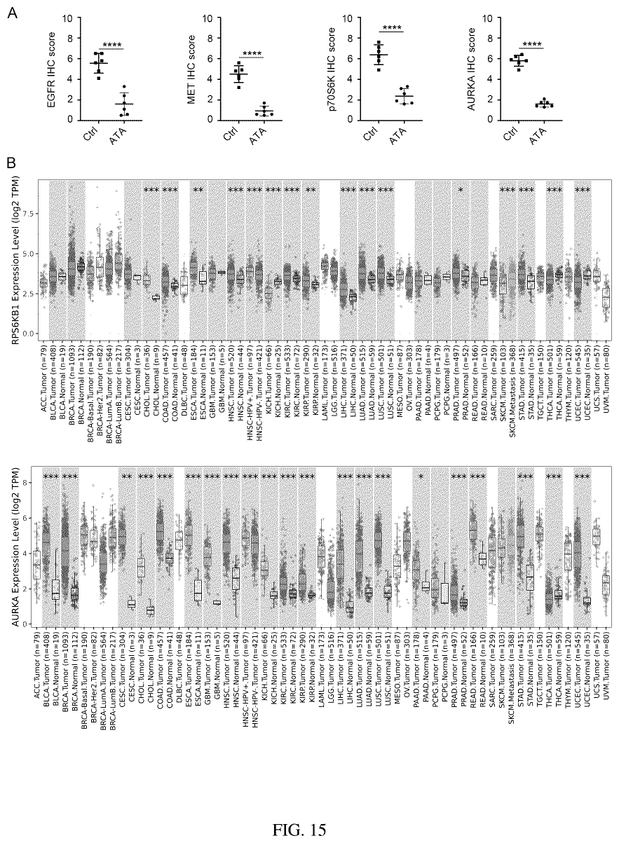

To explore how ATA inhibits the growth of drug-resistant NSCLC cells by decreasing the protein level of p70S6K, RNA sequencing (RNA-seq) analysis was performed on ATA-treated cells and control A549 cells. Analysis of the differences in gene expression between the two groups (as shown in A in FIG. 5) revealed that 1752 genes were significantly up-regulated and 2105 genes were significantly down-regulated, with a fold change greater than 2, P<0.05 (as shown in A in FIG. 12). Subsequently, in order to determine which biological pathways are mainly affected by ATA treatment, gene Ontology (GO) differentially expressed genes. Results show that the biological process that is most significantly up-regulated after ATA treatment is the response to stress, and the biological process that is most significantly down-regulated is the “cell cycle” (as shown in B in FIG. 5). Gene set enrichment analysis (GSEA) also showed that the cell cycle pathway was enriched in cells of control group compared to ATA treated cells (as shown in C in FIG. 5). These findings indicate that ATA inhibits cell cycle progression. Then, the cell cycle distribution of A549 and H1650 cells treated with ATA was examined, and the results showed that ATA increased the proportion of cells in the G1 phase and decreased the proportion of cells in the S phase, meaning that ATA prevented cell migration from G1 phase to S phase during cell cycle progression (as shown in D in FIG. 5 and B in FIG. 12).

To clarify how ATA blocked the cell cycle in the G1/S phase, all genes affected by ATA were analyzed and it was found that ATA treatment affected the expression of 621 cell cycle-related genes. Of these, 132 genes were up-regulated and 489 genes were down-regulated (as shown in E in FIG. 5). Subsequently, 23 significantly up-regulated and 26 significantly down-regulated genes were identified, respectively, with a fold change of greater than 2 and a P-value of less than 0.05 (as shown in C in FIG. 12). Then, one up-regulated gene CDKN1A (p21) and five down-regulated genes CCND3 (cyclin D3), AURKA, BIRC5 (survivin), PLK1, CCNB1 (cyclin B1) were selected, the transcription of which was significantly affected after ATA treatment (as shown in E in FIG. 5).

It was found that ATA treatment significantly increased mRNA and protein levels of p21 in all four drug-resistant NSCLC cell lines (as shown in F and G in FIG. 5). To determine how ATA treatment increases mRNA levels of p21, we examined protein levels of p53 since p53 is a transcriptional activator of p21 in cell cycle progression. Results show that ATA increased protein levels of p53 in A549 and H1975 cells (as shown in G in FIG. 5). These results may be due to ATA exerting stress on cells, thereby creating a “stress” in ATA-treated cells, and p53 may respond to this “stress”, resulting in the accumulation of p53 in stressed cells. p53 then induces transcription of p21, resulting in increased levels of p21 protein that arrests cells in the G1/S phase of the cell cycle. Studies have also found that ATA reduced cyclin D3 mRNA expression and protein levels in all four drug-resistant NSCLC cell lines (as shown in F and G in FIG. 5). Cyclin D3 can form a complex with CDK4 or CDK6 whose activity is required for cell cycle transition from G1 to S phase. These results show that ATA blocks drug-resistant NSCLC cells in the G1/S phase of the cell cycle probably by increasing mRNA and protein levels of p21 and decreasing mRNA and protein levels of cyclin D3.

In FIG. 5, (A) is performing RNA sequencing in duplicate to analyze transcriptome profiles for A549 cells treated with ATA (2 μM) for 48 h. Overall results for FPKM (fragments of transcript sequence per million bases) for cluster analysis using log 10 (FPKM+1) values. A red color indicates a gene with a high expression level, and a blue color indicates a gene with a low expression level. The color range from red to blue represents a log 10 (FPKM+1) value from high to low. (B) is performing gene ontology (GO) analysis to determine the genes most significantly up-regulated or down-regulated in ATA-induced biological processes in A549 cells. (C) is the gene set enrichment analysis (GSEA) of differentially expressed genes in cell cycle-related pathways. (D) is performing cell cycle analysis after propidium iodide (PI) staining and flow cytometer measurements for A549 and H1650 cells treated with ATA (1 or 2 μM) for 48 h. (E) is RNA-seq volcano plots of the ATA-treated group and the A549 cell control group showing cell cycle-related genes affected by ATA. (F) is mRNA levels of p21 and cyclin D3 determined by real-time PCR for A549, H358, H1975, and H1650 cells treated with ATA (1 or 2 μM) for 48 h. Quantification of mRNA levels. *P<0.05, **P<0.01, ***P<0.001, and ****P<0.0001 were based on a two-way analysis of variance and Tukey's multiple comparison test. (G) is protein levels of p21, p53, and cyclin D3 determined by Western blot analysis for A549, H358, H1975, and H1650 cells treated with ATA (1 or 2 μM) for 48 h. (F) is data expressed as the mean±SD of three independent experiments.

In FIG. 12, (A) RNA-seq plots of ATA treated and control groups in A549 cells show significant up- and down-regulation of 1752 and 2105 genes, respectively, with fold changes above 2 and P values<0.05. (B) is A549 and H1650 cells treated with ATA (1 or 2 μM) for 48 h. Cell cycle analysis was performed after propidium iodide (PI) staining and flow cytometry measurements. Quantification of cell cycle distribution of A549 and H1650 cells after ATA treatment is shown. ***P<0.001 and ****P<0.0001 were based on two-way ANOVA followed by Tukey's multiple comparison test. (C) is the heat map of cell cycle-related genes after A549 cells were treated with ATA showing that 23 and 26 genes were significantly up-regulated and down-regulated, respectively, with fold change greater than 2 and P value<0.05. Data are expressed as the mean±SD of three independent experiments.

### Example 6

### ATA Inhibits the Growth of Drug-Resistant NSCLC Cells by Reducing the Protein Level of AURKA

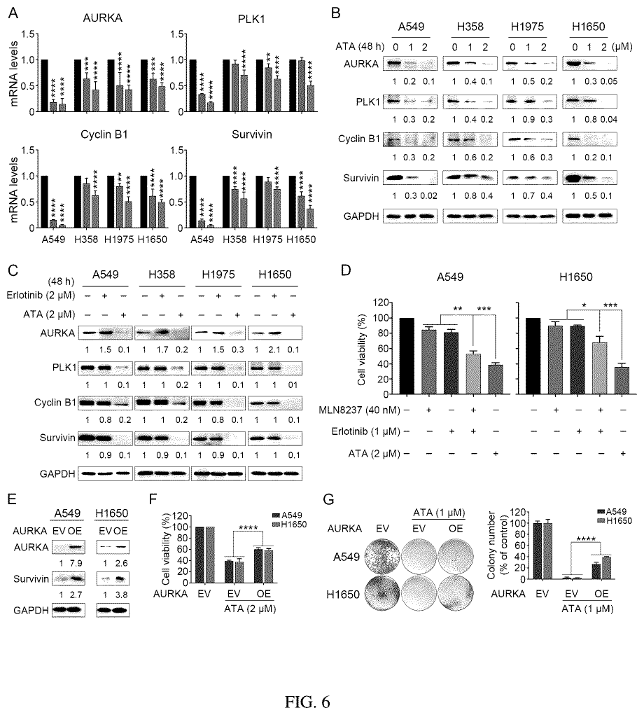

In addition to p21 and cyclin D3, ATA also reduced mRNA and protein levels of Aurora Kinase (AURKA), polo-like kinase (PLK1), cyclin B1, and survivin in drug-resistant NSCLC cells (as shown in A and B in FIG. 6). AURKA may be an effective target for the treatment of drug-resistant NSCLC. It was found that drug-resistant NSCLC cells showed higher AURKA protein levels after Erlotinib treatment (as shown in C in FIG. 6), which may result in resistance of these cells to Erlotinib. In contrast, ATA significantly reduced the protein levels of AURKA, PLK1, cyclin B1, and survivin in all four drug-resistant NSCLC cells (as shown in C in FIG. 6). These findings may explain why ATA is more effective than Erlotinib in inhibiting the growth of these drug-resistant NSCLC cells.

This example study further shows that in A549 and H1650 cells, monotherapy with ATA produced greater growth inhibition than combination therapy with Erlotinib and MLN8237, an AURKA inhibitor (as shown in D in FIG. 6). Subsequently, to further investigate whether ATA can inhibit the growth of drug-resistant NSCLC cells by decreasing AURKA, we evaluated the effect of AURKA overexpression on ATA-mediated inhibition of the growth of drug-resistant NSCLC cells. MTT results show that overexpression of AURKA increased the protein levels of AURKA and survivin (as shown in E in FIG. 6) and partially reversed the growth inhibition of ATA (as shown in F in FIG. 6). Similarly, overexpression of AURKA in A549 and H1650 cells also partially reduced the inhibition of colony formation by ATA (as shown in G in FIG. 6).

Accordingly, the above results show that ATA can inhibit the cell growth of drug-resistant NSCLC by reducing the protein level of AURKA.

In FIG. 6 (A) are mRNA levels of AURKA, PLK1, cyclin B1, and survivin determined by real-time PCR for A549, H358, H1975, and H1650 cells treated with ATA (1 or 2 μM) for 48 h. Quantification of mRNA levels. **P<0.01, ***P<0.001, and ****P<0.0001 were based on a two-way analysis of variance and Tukey's multiple comparison test. (B) is protein levels of AURKA, PLK1, cyclin B1, and survivin determined by Western blot analysis for A549, H358, H1975, and H1650 cells treated with ATA (1 or 2 μM) for 48 h. (C) is protein levels of AURKA, PLK1, cyclin B1, and survivin determined by Western blot analysis for A549, H358, H1975, and H1650 cells treated with Erlotinib (2 μM) or ATA (2 μM) for 48 h. (D) is cell viability measured with MTT assay for A549 and H1650 cells treated with AURKA inhibitor (MLN8237, 40 nM), Erlotinib (1 μM) or ATA (2 μM) for 72 h. *P<0.05, **P<0.01, and ***P<0.001 were one-way analyses of variance and Tukey's multiple comparison test. (E) is transfecting A549 and H1650 cells with empty vector (EV) or AURKA overexpression (AURKA-OE) plasmids. Empty vehicle (EV) served as a control. AURKA and survivin protein levels were determined by Western blot analysis. (F) is cell viability measured by the MTT method for A549 and H1650 cells overexpressing AURKA treated with ATA (2 μM) for 72 h. ****P<0.0001 was based on a one-way analysis of variance followed by Tukey's multiple comparison test. (G) is treating A549 and H1650 cells overexpressing AURKA with ATA (1 μM) for 10 days. The plates were stained with crystal violet. Representative images of three independent experiments are shown. Quantification of colony formation (relative number of colonies compared to that of the control group) is shown. ****P<0.0001 was based on a one-way analysis of variance followed by Tukey's multiple comparison test. Statistical data are expressed as the mean SD of three independent experiments.

### Example 7

### ATA Affects Cell Cycle-Related Proteins by Decreasing Protein Levels of p70S6K

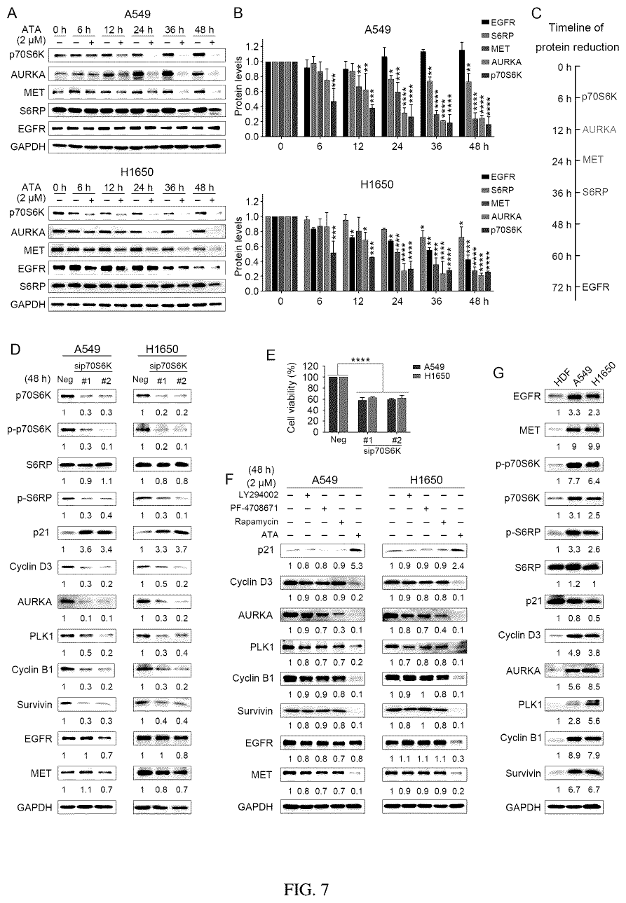

ATA may decrease mRNA and protein levels of cyclin D3 by decreasing protein levels of p70S6K. To determine which protein was first affected by ATA, A549, and H1650 cells were treated with 2 μM ATA for various times. Western blot results showed that ATA reduces the levels of each protein in the following chronological order: 6 hours, p70S6K decreased; 12 hours, AURKA decreased; 24 hours, MET decreased; 36 hours, S6RP decreased; 48-72 hours, EGFR decreased (as shown in A to C in FIG. 7). These findings indicate that ATA reduces first the p70S6K protein and then the protein levels of cell cycle-related proteins (such as AURKA) and receptor proteins (such as EGFR and MET).

Subsequently, in order to determine whether ATA affects cell cycle-related proteins by decreasing protein levels of p70S6K, 70S6K siRNA was used to silence p70S6K expression. It was found that p70S6K siRNA significantly decreased protein levels of p70S6K, and more importantly, decreased expression of p70S6K reduced phosphorylation of p70S6K and S6RP in A549 and H1650 cells (as shown in D in FIG. 7). In addition, p70S6K siRNA increased protein levels of p21 and decreased protein levels of cyclin D3, AURKA, PLK1, cyclin B1 and survivin. However, p70S6K siRNA had less effect on EGFR and MET (as shown in D in FIG. 7). Finally, the effect of p70S6K siRNA on growth inhibition of A549 and H1650 cells was assessed and MTT results showed that p70S6K siRNA significantly reduced cell growth by 40% in both cell lines (as shown in E in FIG. 7).

Further, the effects of PI3K inhibitors (LY294002), p70S6K inhibitors (PF-4708671), mTOR inhibitors (Rapamycin), and ATA on these cell cycle-related proteins, EGFR, and MET were compared. Western blot results showed that LY294002, PF-4708671, and rapamycin were much less effective than ATA in increasing protein levels of p21 and decreasing protein levels of cyclin D3, AURKA, PLK1, cyclin B1, survivin, EGFR, and MET (as shown in F in FIG. 7).

In addition, the expression levels of these proteins were compared between normal fibroblasts (HDF) and lung cancer cells (A549 and H1650). Results show that lung cancer cells expressed 2.3-14.5-fold higher levels of EGFR, MET, p-p70S6K, p70S6K, p-S6RP, cyclin D3, AURKA, PLK1, cyclin B1, similar levels of S6RP, and lower levels of p21 compared to normal cells (as shown in G in FIG. 7 and FIG. 13). These findings may explain why ATA has better growth inhibition in cancer cells than in normal cells.

Accordingly, the above results show that ATA inhibits the growth of drug-resistant NSCLC cells by decreasing the protein level of p70S6K and then affecting other cell cycle-related proteins.

In FIGS. 7, A549 and H1650 cells were treated with ATA (2 μM) for 6, 12, 24, 36, and 48 h, and the protein levels of p70S6K, AURKA, MET, S6RP, and EGFR at each time point were determined by Western blot analysis as shown in (A), and the quantified protein levels are shown in (B). (C) shows the timeline for protein reduction during ATA treatment. (D) is protein levels of p70S6K, p-p70S6K, S6RP, p-S6RP, p21, cyclin D3, AURKA, PLK1, cyclin B1, survivin, EGFR, and MET determined by Western blot analysis for A549 and H1650 cells treated with 10 nM p70S6K siRNA and siRNA negative control for 48 h. (E) is cell viability measured by MTT test for A549 and H1650 cells treated with 10 nM p70S6K siRNA and the negative control siRNA for 48 h. ****P<0.0001 was based on a one-way analysis of variance followed by Tukey's multiple comparison test. (F) It is protein levels of p21, cyclin D3, AURKA, PLK1, cyclin B1, survivin, EGFR, and MET determined by Western blot analysis for A549 and H1650 cells treated with LY294002 (2 μM), PF-4708671 (2 μM), rapamycin (2 μM) and ATA (2 μM) for 48 h. (G) is expression levels of EGFR, MET, p-p70S6K, p70S6K, p-S6RP, S6RP, p21, cyclin D3, AURKA, PLK1, cyclin B1 and survivin in fibroblast HDF, A549 and H1650 cells determined by Western blot analysis, respectively. Statistical data are expressed as the mean SD of three independent experiments.

FIG. 13 shows the expression levels of EGFR, MET, p-p70S6K, p70S6K, p-S6RP, S6RP, p21, cyclin D3, AURKA, PLK1, cyclin B1, and survivin in HDF, A549 and H1650 cells after analysis and quantification by Western blot.

### Example 8

### ATA Inhibits the Growth of Drug-Resistant NSCLC-Derived Xenograft Tumors in Mice

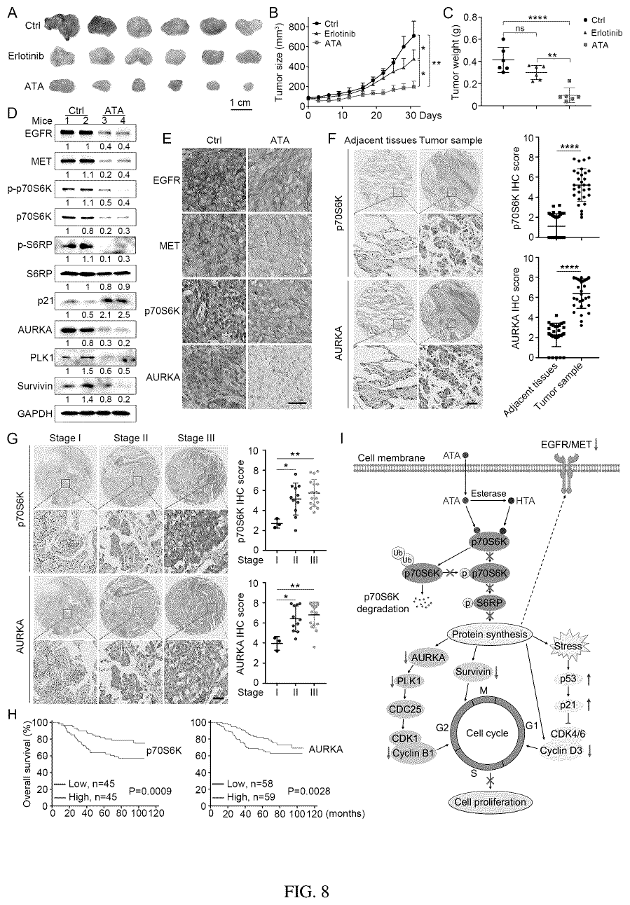

Based on the above examples it has been demonstrated that ATA can inhibit the growth of drug-resistant NSCLC cells in vitro. To determine if ATA could also produce the same effect in vivo, this example injected A549 cells subcutaneously into nude mice to form tumor xenografts. Mice were randomly assigned to three groups. When the size of each tumor increased to a volume of approximately 100 mm³, mice were injected intraperitoneally with vehicle control, ATA (25 mg/kg), or Erlotinib (25 mg/kg) every 3 days for 31 days. The tumor size and body weight of each mouse were measured every 3 days until the end of the animal experiment on day 31. The results showed that at day 31, Erlotinib reduced tumor size by 32.5% compared to the control group, but it did not significantly reduce tumor weight. In contrast, ATA showed higher tumor growth inhibitory potency than Erlotinib, reducing tumor size by 72.1% and tumor weight by 77.4% (as shown in A TO C in FIG. 8). In addition, there was no difference in the weight of nude mice between the three groups (as shown in FIG. 14), and no mice died during the treatment period. These data indicate that ATA strongly inhibited the growth of A549 cell-derived xenograft tumors and produced no significant toxicity in nude mice.

Subsequently, relevant protein levels in A549-derived xenograft tumor tissue were tested and the results showed that protein levels of EGFR, MET, p-p70S6K, p70S6K, p-S6RP, AURKA, PLK1, and survivin were greatly reduced and p21 protein levels were increased more than 2-fold after ATA treatment (as shown in D in FIG. 8). In addition, the levels of EGFR, MET, p70S6K and AURKA proteins in A549 derived xenografts were examined by IHC analysis. The staining results showed that the levels of these proteins in tumors also decreased significantly after ATA treatment (as shown in E in FIG. 8). All these data illustrate that ATA inhibited the growth of A549-derived xenograft tumors and decreased the levels of EGFR, MET, p70S6K and AURKA proteins in tumor tissues.

To further determine the clinical significance of p70S6K and AURKA expression in lung adenocarcinoma patients, two sets of tissue chips containing 30 lung cancer samples and 30 adjacent tissues were also purchased during the course of this example. These cancer samples include stage I, II, and III tumor tissues from lung adenocarcinoma patients. IHC staining was performed on these tissue samples and the intensity of staining was expressed using the IHC score. For each sample, the IHC score ranges from 0-8 points, consisting of a proportional score (0=0% proportion; 1=1% proportion; 2=10% proportion; 3=33% proportion; 4=66% proportion; 5=100% proportion) and an intensity score (0=negative; 1=weak; 2=medium; 3=strong). Results show that p70S6K and AURKA proteins are expressed at much higher levels in tumor samples compared to adjacent tissues. Specifically, the IHC score for p70S6K was 4.7-fold higher in tumor samples and the IHC score for AURKA was 2.8-fold higher in tumor samples (as shown in F in FIG. 8). Furthermore, in many different types of cancer, p70S6K and AURKA are also highly expressed in cancer samples compared to normal samples (as shown in FIG. 15).

It was also found through the study of this example that the staining intensity of p70S6K and AURKA was significantly higher in stage II and III tumor samples than in stage I tumor samples (as shown in G in FIG. 8). Finally, UALCAN analysis showed that high expression of p70S6K and AURKA correlated with shorter overall survival in lung adenocarcinoma patients (as shown in H in FIG. 8), indicating that high expression of p70S6K and AURKA proteins was clinically relevant for the progression of lung adenocarcinoma.

FIG. 8 (A) is that 4 million A549 cells were introduced subcutaneously into nude mice to form tumor xenografts. Animals were injected intraperitoneally with 25 mg/kg ATA, 25 mg/kg Erlotinib, or vehicle control once every 3 days for 31 days, and tumor volumes (mm³) were measured every 3 days. Representative images of tumor xenografts from the control, ATA-treated, and Erlotinib-treated groups were obtained at the end of the animal experiment. Scale bar, 1 cm. (B) is tumor volume data from 6 mice/group. Data are expressed as mean SD. *P<0.05 and **P<0.01 were based on a two-tailed unpaired t-test. (C) is tumor weight data from 6 mice/group. Data are expressed as mean SD. **P<0.01 and ****P<0.0001 were based on a one-way analysis of variance and Tukey's multiple comparison test. (D) is lysates of individual xenograft tumors from control and ATA-treated groups collected accordingly. EGFR, MET, p-p70S6K, p70S6K, p-S6RP, S6RP, p21, AURKA, PLK1, and survivin protein levels were detected by Western blot analysis. Immunoblot samples were from two independent tumors per group. (E) is representative IHC image: EGFR, MET, p70S6K, and AURKA from mouse A549 xenograft tumors treated with solvent control or ATA. Magnification is 20×. Scale bar, 50 μm. (F) are representative images of IHC staining of p70S6K and AURKA for patient-derived lung tumors (n=30) and adjacent tissues (n=30). Mean levels of p70S6K and AURKA in patient-derived tumors and adjacent tissues as quantified by IHC staining. Images were magnified 5× and 20×. Scale bar, 100 μm. ****P<0.0001 was based on a two-tailed analysis and an unpaired t-test. (G) are representative IHC staining images of p70S6K and AURKA, patient-derived lung tumors from stage I (n=3), stage II (n=11), and III (n=16). Mean levels of p70S6K and AURKA in tumor-derived tumors from patients in stages I, II, and III, as quantified by IHC staining. Images were taken at 5× and 20× magnification. Scale bar, 100 μm. *P<0.05 and **P<0.01 were based on a one-way analysis of variance followed by Tukey's multiple comparison test. (H) is plotting the overall survival of lung adenocarcinoma patients with different expression levels of p70S6K (GSE11969) and AURKA (GSE13213) using UALCAN analysis. The website with the overall survival analysis tool is http://genomics.jefferson.edu/proggene/index.php. (I) is a proposed mechanism for ATA targeting p70S6K to inhibit the synthesis of cell cycle-related proteins and subsequently inhibit the proliferation of drug-resistant NSCLC cells.

FIG. 14 is an injection of 25 mg/kg Erlotinib, ATA, or vehicle control by intraperitoneal administration into nude mice with A549-derived xenograft tumors. The body weights of each mouse were recorded every 3 days for 31 days. Mean body weights were obtained from six mice in each group and expressed as mean±SD.

The clinical data of p70S6K (RPS6KB1) and AURKA in FIG. 15 are from the website (http://timer.cistrome.org/).

In view of the above, the present disclosure finds that ATA has good efficacy in vitro and in vivo in the treatment of primary or acquired drug resistance of NSCLC to EGFR TKIs. The results of the disclosed studies indicate that ATA is effective in inhibiting the growth, colony formation, spheroid formation, migration, and invasion of drug-resistant NSCLC cells. More importantly, ATA strongly inhibited the growth of xenograft tumors of A549 cells in nude mice. Mechanistic studies suggest that ATA may overcome the primary and acquired drug resistance of NSCLC to EGFR TKIs by targeting p70S6K and its downstream signaling molecules. The first set of targets for ATA includes p70S6K and its substrate S6RP. ATA and its metabolite HTA bind to the ATP binding site of p70S6K thereby preventing its phosphorylation. ATA also increases ubiquitination-mediated degradation of p70S6K, resulting in decreased protein levels. Inhibition of p70S6K by ATA further prevents activation of S6RPs critical for protein synthesis. The second group includes p53, p21, and survivin. ATA increases the level of p53 which increases the transcription of p21 and prevents cells from entering the S phase from G1. As a transcription factor, p53 can also inhibit survivin transcription, which may in part result in a decrease in its protein levels. Survivin has two functions: first, it acts as an anti-apoptotic protein, and second, it plays a positive role in mitosis. The third group contains several proteins essential for cell cycle progression, such as cyclin D3, AURKA, PLK1, and cyclin B1. ATA can decrease the protein levels of these cell cycle-related proteins by decreasing p70S6K. This may be because many of the proteins involved in cell cycle control are newly produced at every stage of the cell cycle, and all of these new proteins cannot be synthesized when p70S6K is inhibited by ATA. The last group includes the two members of the receptor tyrosine kinases, EGFR and MET.

Although ATA can reduce the levels of many proteins, the main target of ATA may be p70S6K, which plays an important role in the regulation of protein synthesis, from the results of decreased timeline of protein and silencing p70S6K gene expression. Cancer cells grow faster than normal cells and therefore require high levels of protein synthesis. The results of the present disclosure show that compared to normal fibroblasts, many ATA down-regulated proteins including EGFR, MET, p70S6K, p-S6RP, cyclin D3, AURKA, PLK1, cyclin B1, and survivin are highly expressed in lung cancer cells. From clinical samples, it was also observed that the protein levels of p70S6K and AURKA were much higher in tumor samples from NSCLC patients than in their adjacent tissues. Many studies have also shown that p70S6K and AURKA are overexpressed or activated in many types of cancer, suggesting that these two kinases may contribute to tumorigenesis.

In addition, p70S6K is involved in tumor metastasis and drug resistance, and overexpression or activation of AURKA is also involved in resistance to EGFR TKIs. Clinical sample results also showed higher expression of p70S6K and AURKA in NSCLC stage II and III stage tumor samples compared to stage I tumor samples. Furthermore, UALCAN analysis showed that high expression of p70S6K and AURKA correlated with poor prognosis in lung adenocarcinoma patients. Therefore, targeting p70S6K and AURKA may be beneficial in the treatment of NSCLC patients with primary or acquired drug resistance to EGFR TKIs.

The results of the studies in the present disclosure also show that compared with PI3K inhibitor LY294002, p70S6K inhibitor PF-4708671 and mTOR inhibitor rapamycin, ATA has better inhibitory effect on cell growth and protein synthesis by reducing the protein level of p70S6K. Furthermore, the present disclosure also found that drug-resistant NSCLC cells increase protein levels of p70S6K and AURKA after treatment with Erlotinib. Increased p70S6K and AURKA may result in NSCLC drug resistance to Erlotinib. Relevant study results showed that ATA reduces protein levels of p70S6K and AURKA, resulting in sustained and irreversible inhibition of kinase activity. This effect explains why ATA is better than Erlotinib in inhibiting the growth of drug-resistant NSCLC cells.

In the studies in the present disclosure, it was also found that in addition to p70S6K and AURKA, ATA can also reduce the levels of multiple proteins in drug-resistant NSCLC cells, including PLK1, cyclin B1, survivin, EGFR, and MET. These multi-target effects of ATA should enable ATA to inhibit multiple signaling pathways and overcome drug resistance of NSCLC to EGFR TKIs.

In summary, the above studies collectively indicate that ATA may be an effective anticancer agent to treat drug-resistant NSCLC by degrading p70S6K, AURKA, and other cell cycle-related proteins.

The foregoing is merely illustrative of alternative embodiments of the present disclosure and is not intended to limit the present disclosure. Various modifications and variations of the present disclosure will be apparent to those skilled in the art. All changes, equivalents, improvements, etc. that come within the spirit and scope of the disclosure are to be embraced within their scope.

### INDUSTRIAL APPLICABILITY

The present disclosure provides a use of acetyltanshinone IIA in the preparation of a medicament for treating lung cancer and medicament for treating lung cancer, which can antagonize primary and acquired drug resistance of NSCLC cells to epidermal growth factor receptor, i.e., tyrosine kinase inhibitor (EGFR TKIs), by using a small-molecule compound acetyltanshinone IIA. The medicament containing acetyltanshinone IIA is expected to be developed as a multi-target anticancer agent for the treatment of TKI-resistant NSCLC with excellent practicality.

1. Use of acetyltanshinone IIA in the preparation of a medicament for treating lung cancer, wherein the acetyltanshinone IIA is used for the preparation of a medicament for treating non-small cell lung cancer caused by KRAS activating mutation or PTEN deletion.

2. Use of acetyltanshinone IIA in the preparation of a lung cancer cell growth inhibitor, wherein the acetyltanshinone IIA is used for the preparation of a non-small cell lung cancer cell growth inhibitor.

3. The use of claim 2, wherein the acetyltanshinone IIA is used for the preparation of an H358 cell growth inhibitor, an H1975 cell growth inhibitor, and/or an H1650 cell growth inhibitor.

4. Use of acetyltanshinone IIA in the preparation of a protein synthesis inhibitor.

5. The use of claim 4, wherein the protein synthesis inhibitor comprises an inhibitor of cell cycle-related protein synthesis.

6. The use of claim 4, wherein the protein corresponding to the protein synthesis inhibitor comprises at least one of p70S6K, cyclin D3, AURKA, PLK1, cyclin B1, survivin, EGFR, and MET.

7. Use of acetyltanshinone IIA in the preparation of an inhibitor for phosphorylation level of signaling molecules downstream of proteins.

8. The use of claim 7, wherein the acetyltanshinone IIA is used for the preparation of a p70S6K and/or S6RP phosphorylation inhibitor.

9. Use of acetyltanshinone IIA in the preparation of a p21 transcriptional activator or p53 promoter.

10. A medicament for treating lung cancer, wherein the composition of the medicament contains acetyltanshinone IIA, and the medicament is for treating non-small cell lung cancer caused by KRAS activating mutation or PTEN deletion.

11. The medicament of claim 10, wherein the composition of the medicament further comprises a lung cancer cell growth inhibitor, a protein synthesis inhibitor, an inhibitor for phosphorylation level of signaling molecules downstream of proteins, a p21 transcriptional activator and a p53 promoter containing acetyltanshinone IIA.

12. Use of the medicament of claim 10 for the treatment of a disease associated with non-small cell lung cancer caused by KRAS activating mutation or PTEN deletion.

13. Use of the medicament of claim 11 for the treatment of a disease associated with non-small cell lung cancer caused by KRAS activating mutation or PTEN deletion.

14. A method for treating a disease associated with lung cancer in a subject, comprising: administering to a subject in need thereof the medicament of claim 10, the disease associated with lung cancer is non-small cell lung cancer caused by KRAS activating mutation or PTEN deletion.

15. A method for treating a disease associated with lung cancer in a subject, comprising: administering to a subject in need thereof the medicament of claim 11, the disease associated with lung cancer is non-small cell lung cancer caused by KRAS activating mutation or PTEN deletion.

16. The use of claim 12, wherein the non-small cell lung cancer comprises: adenocarcinoma, squamous cell carcinoma, and large cell carcinoma.

17. The use of claim 13, wherein the non-small cell lung cancer comprises: adenocarcinoma, squamous cell carcinoma, and large cell carcinoma.

18. The method of claim 14, wherein the non-small cell lung cancer comprises: adenocarcinoma, squamous cell carcinoma, and large cell carcinoma.

19. The method of claim 15, wherein the non-small cell lung cancer comprises: adenocarcinoma, squamous cell carcinoma, and large cell carcinoma.


SEQUENCE LISTING (22 sequences)

SEQ ID NO: 1 - DNA, 21 bp, synthetic construct
  source 1..21: mol_type = other DNA; note = primer
gaggtccaaaacgtgttctcg

SEQ ID NO: 2 - DNA, 22 bp, synthetic construct
  source 1..22: mol_type = other DNA; note = primer
acaggatgaggtacactggttg

SEQ ID NO: 3 - DNA, 21 bp, synthetic construct
  source 1..21: mol_type = other DNA; note = primer
aggaccaccgcatctctacat

SEQ ID NO: 4 - DNA, 21 bp, synthetic construct
  source 1..21: mol_type = other DNA; note = primer
aagtctggctcgttctcagtg

SEQ ID NO: 5 - DNA, 22 bp, synthetic construct
  source 1..22: mol_type = other DNA; note = primer
aactttcgcctgagcctatttt

SEQ ID NO: 6 - DNA, 21 bp, synthetic construct
  source 1..21: mol_type = other DNA; note = primer
ttggtctgactgcttgctctt

SEQ ID NO: 7 - DNA, 19 bp, synthetic construct
  source 1..19: mol_type = other DNA; note = primer
tacccgccatccatgatcg

SEQ ID NO: 8 - DNA, 19 bp, synthetic construct
  source 1..19: mol_type = other DNA; note = primer
aggcagtccacttcagtgc

SEQ ID NO: 9 - DNA, 22 bp, synthetic construct
  source 1..22: mol_type = other DNA; note = primer
cgatggaacttcgactttgtca

SEQ ID NO: 10 - DNA, 21 bp, synthetic construct
  source 1..21: mol_type = other DNA; note = primer
gcacaagggtacaagacagtg

SEQ ID NO: 11 - DNA, 21 bp, synthetic construct
  source 1..21: mol_type = other DNA; note = primer
aggcacgagtaacaagctcac

SEQ ID NO: 12 - DNA, 21 bp, synthetic construct
  source 1..21: mol_type = other DNA; note = primer
atgaggacataaccagccacc

SEQ ID NO: 13 - DNA, 20 bp, synthetic construct
  source 1..20: mol_type = other DNA; note = primer
gtcagtggtggacctgacct

SEQ ID NO: 14 - DNA, 20 bp, synthetic construct
  source 1..20: mol_type = other DNA; note = primer
aaaggtggaggagtgggtgt

SEQ ID NO: 15 - DNA, 22 bp, synthetic construct
  source 1..22: mol_type = other DNA; note = primer
agcaatggggagtgtaaagagg

SEQ ID NO: 16 - DNA, 21 bp, synthetic construct
  source 1..21: mol_type = other DNA; note = primer
cccagtcttgtactcagcaac

SEQ ID NO: 17 - DNA, 21 bp, synthetic construct
  source 1..21: mol_type = other DNA; note = primer
aaagagatcccggaggtccta

SEQ ID NO: 18 - DNA, 22 bp, synthetic construct
  source 1..22: mol_type = other DNA; note = primer
ggctgcggtgaatggatatttc

SEQ ID NO: 19 - DNA, 21 bp, synthetic construct
  source 1..21: mol_type = other DNA; note = primer
agaacttctggctcgaaaggt

SEQ ID NO: 20 - DNA, 21 bp, synthetic construct
  source 1..21: mol_type = other DNA; note = primer
cgacaggtgtctgacgtgtaa

SEQ ID NO: 21 - RNA, 17 nt, synthetic construct
  source 1..17: mol_type = other RNA; note = siRNA
catggaacattgagaaa

SEQ ID NO: 22 - RNA, 18 nt, synthetic construct
  source 1..18: mol_type = other RNA; note = siRNA
ggttttcaagtacgaaaa